(19) United States
(12) Patent Application Publication
(10) Pub. No.: US 2020/0316178 A1
(43) Pub. Date: Oct. 8, 2020

(54) FORMULATIONS COMPRISING GLUCOCEREBROSIDASE AND ISOFAGOMINE

(71) Applicant: SHIRE HUMAN GENETIC THERAPIES, INC., Lexington, MA (US)

(72) Inventors: Yung Hee PARK, Arlington, MA (US); Nancy CHEN, Winchester, MA (US); Jun HU, Sudbury, MA (US); Muthuraman MEIYAPPAN, Lexington, MA (US); Thomas Allen MILLER, Wakefield, MA (US)

(73) Assignee: SHIRE HUMAN GENETIC THERAPIES, INC., Lexington, MA (US)

(21) Appl. No.: 16/759,201

(22) PCT Filed: Oct. 25, 2018
(86) PCT No.: PCT/US2018/057575

§ 371 Date: Apr. 24, 2020

Related U.S. Application Data
(60) Provisional application No. 62/577,429, filed on Oct. 26, 2017.

(51) Int. Cl. A61K 38/47; A61K 31/445; A61K 47/02; A61K 47/12; A61K 47/26

(52) U.S. Cl. CPC A61K 38/47; A61K 31/445; A61K 9/0019; A61K 47/12; A61K 47/26; A61K 47/02

(57) ABSTRACT

The invention provides a composition of glucocerebrosidase, such as velaglucerase alfa, and isofagomine, in a molar ratio of at least about 1:2.5. Also provided is a use of the composition for treatment of a disorder related to a dysfunction in a GCase pathway. The disorder could be a lysosomal storage disease, such as Gaucher disease, Fabry disease, Pompe disease, a mucopolysaccharidoses, or multiple system atrophy. The disorder could also be a neurodegenerative disorder, such as Parkinson disease, Alzheimer's disease, or Lewy body dementia. The composition can have 0.5 to 5.0 mg/kg of glucocerebrosidase and isofagomine in at least about a 3-fold molar excess to the glucocerebrosidase. The composition can be administered intravenously or subcutaneously.

### CROSS-REFERENCE TO RELATED APPLICATIONS

This application claims priority to U.S. Provisional Application No. 62/577,429, filed on Oct. 26, 2017, the disclosure of which is herein incorporated by reference in its entirety.

### BACKGROUND OF THE INVENTION

Glucocerebrosidase (GCB) is a protein drug that may be used to treat Gaucher disease, an autosomal recessive lysosomal storage disorder characterized by a deficiency in (GCB).

Gaucher disease is an autosomal recessive disorder caused by mutations in the GBA gene, which results in a deficiency of the lysosomal enzyme beta-glucocerebrosidase. Glucocerebrosidase catalyzes the conversion of the sphingolipid glucocerebroside into glucose and ceramide. The enzymatic deficiency causes an accumulation of glucocerebroside primarily in the lysosomal compartment of macrophages, giving rise to foam cells or “Gaucher cells”. In Gaucher disease, various forms of mutant GCase have reduced, little, or no glucosylceramide cleavage activity, depending upon the mutated amino acid or amino acids. The severity of this disorder is correlated with relative levels of residual enzyme activity and the resulting extent of accumulation of the substrate.

GCB is a lysosomal enzyme that hydrolyzes the glycolipid glucocerebroside that is formed after degradation of glycosphingolipids in the membranes of white blood cells and red blood cells. The deficiency in this enzyme causes glucocerebroside to accumulate in large quantities in the lysosomes of phagocytic cells located in the liver, spleen, and bone marrow of Gaucher patients. Accumulation of these molecules causes a range of clinical manifestations including splenomegaly, hepatomegaly, skeletal disorder, thrombocytopenia and anemia. (Beutler et al. “Gaucher disease” The Metabolic and Molecular Bases of Inherited Disease (McGraw-Hill, Inc, New York, 1995, pp. 2625-2639.)

Velaglucerase alfa is a form of GCB used to treat Gaucher disease. VPRIV is a formulation that contains velaglucerase alfa. Velaglucerase alfa catalyzes the hydrolysis of glucocerebroside, reducing the amount of accumulated glucocerebroside. In clinical trials VPRIV reduced spleen and liver size, and improved anemia and thrombocytopenia.

VPRIV and velaglucerase alfa, and other similar drug products that contain a protein are stored in liquid or lyophilized, i.e., freeze-dried, form. A lyophilized drug product is often reconstituted by adding a suitable administration diluent just prior to patient use. There can be a reduction in the amount of velaglucerase alfa or GCB in liquid or lyophilized form as a result of physical instabilities, including denaturation and aggregation, as well as chemical instabilities, including, for example, hydrolysis, deamidation, and oxidation.

There is a need for improved formulations with improved stability of GCB, VPRIV, or velaglucerase alfa, especially those that are suitable for subcutaneous (SC) administration. GCB has a solubility limit of less than 30 mg/mL at room temperature over 24 hours. A convenient volume for a SC injection product is typically 2.5 mL or less. This necessitates having a formulation that can be concentrated to a high enough level to administer a therapeutically adequate dose. Additionally, the formulations would ideally have appropriate storage stability at room temperature or under refrigerated conditions.

There is also a need for formulations for SC administration that have improved bioavailability of GCB, VPRIV, or velaglucerase alfa. The current VPRIV formulation, which is administered intravenously (IV), provides approximately 1% of serum bioavailability. Subcutaneous (SC) administration would be unlikely to provide the equivalent tissue exposure as that of an IV administration. GCB has a serum half-life of less than 15 minutes as an IV administered drug. Improved serum stability would allow more SC-administered GCB to disperse out of the SC compartment and into the systemic circulation. Enhanced serum stability would also enable the maintenance of high circulating GCB concentrations, thus enabling more GCB to be taken up by monocytes, macrophages, and tissue-resident histiocytes.

### SUMMARY OF THE INVENTION

In one aspect is provided a composition comprising a glucocerebrosidase (GCB) and an isofagomine (IFG) in a molar ratio of 1:1 or at least about 1:>1 (e.g., 1:x, where x is greater than 1). In some embodiments, the GCB is velaglucerase alfa. Velaglucerase alfa is a recombinantly-produced enzyme with the same amino acid sequence as naturally-occurring human GCB produced in a human cell line, and is an especially suitable form of GCB for practicing the invention. In some embodiments, the pH of the composition is about 6.0. In some embodiments, the pH of the composition is about 6.5. In some embodiments, the pH of the composition is about 7.0. In some embodiments, the molar ratio of the GCB to the IFG is from about 1:1 to about 1:30. In some embodiments, the molar ratio of the GCB to the IFG is from about 1:1 to about 1:10. In some embodiments, the molar ratio of the GCB to the IFG is from about 1:1 to about 1:5. In some embodiments, the molar ratio of the GCB to the IFG is from about 1:2 to about 1:10. In some embodiments, the molar ratio of the GCB to the IFG is from about 1:2.5 to about 1:10. In some embodiments, the molar ratio of the GCB to the IFG is from about 1:2.5 to about 1:5. In some embodiments, the molar ratio of the GCB to the IFG is from about 1:10 to about 1:30. In some embodiments, the molar ratio of the GCB to the IFG is from about 1:30 to about 1:100. In some embodiments, the molar ratio of the GCB to the IFG is about 1:2.5 to about 1:3.5. In some embodiments, the molar ratio of the GCB to the IFG is about 1:3.0. In some embodiments, the molar ratio of the GCB to the IFG is 1:3.0, which is especially suitable for practicing the invention.

In some embodiments, the composition is at a temperature of at least 20° C. In some embodiments, the composition is at a temperature of 0° C. to 20° C. In some embodiments, the composition is at a temperature of less than 0° C. In some embodiments, the composition is an aqueous solution. In some embodiments, the composition is a lyophilizate.

In some embodiments, the composition further comprises a pharmaceutically acceptable excipient, a pharmaceutically acceptable salt, or both a pharmaceutically acceptable excipient and a pharmaceutically acceptable salt.

In some embodiments, the IFG is isofagomine tartrate (IFGT). In some embodiments, the isofagomine tartrate is isofagomine D-tartrate. IFGT, and in particular isofagomine D-tartrate, are especially suitable salts of IFG for practicing the invention. Isofagomine tartrate can advantageously increase GCB activity in the serum above the upper limit normally achieved with a subcutaneous dose of 2.5 mg/kg. Accordingly, GCB co-formulated with IFGT can provide serum bioavailability that allows for subcutaneous administration, in particular when at a molar ratio of at least 1:3.0 GCB: IFGT. IFGT co-formulation also increases the overall enzyme activity of GCB. In some embodiments, the IFG is other than isofagomine tartrate. In some embodiments, the composition is a liquid. In some embodiments, the composition further comprises an antioxidant. In some embodiments, the composition further comprises a carbohydrate. In some embodiments, the composition further comprises a surfactant. In some embodiments, the composition comprises 45-120 mg/mL of velaglucerase alfa and 0.2 to 1.8 mg/mL isofagomine D-tartrate. In some embodiments, the composition comprises 60 mg/mL of velaglucerase alfa and 0.9 mg/mL isofagomine D-tartrate.

In some embodiments, the composition further comprises citrate or phosphate and polysorbate 20 (e.g., 50 mM sodium citrate or sodium phosphate, and 0.01% polysorbate 20). In some embodiments, the composition further comprises 5-20 mM sodium citrate and 0.01% polysorbate-20. In some embodiments, the composition further comprises 10 mM sodium citrate and 0.01% polysorbate-20. In some embodiments, the composition further comprises 5-20 mM sodium phosphate and 0.01% polysorbate-20. In some embodiments, the composition further comprises 10 mM sodium phosphate and 0.01% polysorbate-20. In some embodiments, the composition further comprises 5-20 mM sodium citrate and 0.01% (w/v) polysorbate-20. In some embodiments, the composition further comprises 10 mM sodium citrate and 0.01% (w/v) polysorbate-20. In some embodiments, the composition further comprises 5-20 mM sodium phosphate and 0.01% (w/v) polysorbate-20. In some embodiments, the composition further comprises 10 mM sodium phosphate and 0.01% (w/v) polysorbate-20. In some embodiments, the composition further comprises 5-20 mM sodium citrate and 0.01% (v/v) polysorbate-20. In some embodiments, the composition further comprises 10 mM sodium citrate and 0.01% (v/v) polysorbate-20. In some embodiments, the composition further comprises 5-20 mM sodium phosphate and 0.01% (v/v) polysorbate-20. In some embodiments, the composition further comprises 10 mM sodium phosphate and 0.01% (v/v) polysorbate-20. In some embodiments, the composition is at about pH 6.0. In some embodiments, the composition is at pH 6.0.

In another aspect is provided a container comprising any of the compositions described herein. In some embodiments, the container is selected from the group consisting of a prefilled syringe, a vial, or ampoule.

In another aspect is provided a method of preparing any of the compositions described herein. The method comprises dissolving the IFG (e.g., in water), adjusting the pH to about 6.0, and adding the GCB to yield the composition. In some embodiments, the method further comprises lyophilizing the IFG before adding GCB. In some embodiments, the method further comprises adding polysorbate 20 to 0.01%. In some embodiments, the method further comprises adding polysorbate 20 to 0.01% (w/v). In some embodiments, the method further comprises adding polysorbate 20 to 0.01% (v/v). In some embodiments, the method further comprises filtering the composition through a 0.22 μm membrane. In some embodiments, the IFG is present in an amount sufficient to maintain the stability of the GCB in the composition. In some embodiments, the IFG is present in an amount sufficient to maintain the stability of the GCB in the composition for at least three days at 0-50° C. In some embodiments, the IFG is present in an amount sufficient to maintain the stability of the GCB in the composition for at least 6 months at 0-40° C.

In another aspect is provided a method of treating a disorder related to a dysfunction in a GCase pathway comprising administering any of the compositions described herein. In some embodiments, the method is effective to treat the disorder. In some embodiments, the composition is administered intravenously or subcutaneously. In some embodiments, the composition is administered subcutaneously, e.g., by subcutaneous injection, which is especially suitable for practicing the invention. In some embodiments, the composition is administered twice weekly, once weekly, less often than once weekly, or once every other week. Typically, the compositions described herein are administered subcutaneously by injection either once or twice a week, or once every other week. Compositions described herein (in particular, formulations with IFGT) administered subcutaneously can provide significantly greater serum exposure compared to comparable intravenous doses of GCB alone. Greater serum bioavailability advantageously allows a reduction in the number of subcutaneous injections that need to be administered to a subject. For example, fewer injections need to be administered per treatment to achieve a therapeutically effective amount and/or the time interval between subcutaneous injections can be extended.

In another aspect, the compositions described herein are for use in therapy. In one embodiment, the compositions described herein are for use in a method of treating a disorder related to a dysfunction in a GCase pathway as disclosed herein. In another embodiment, the compositions described herein are for use in the manufacture of a medicament for treating a disorder related to a dysfunction in a GCase pathway, e.g. by the methods disclosed herein. In some embodiments, the composition is administered intravenously or subcutaneously. In some embodiments, the composition is administered subcutaneously, e.g., by subcutaneous injection. In some embodiments, the composition is administered twice weekly, once weekly, less often than once weekly, or once every other week. Typically, the compositions described herein are administered subcutaneously by injection either once or twice a week, or once every other week.

In some embodiments, the disorder comprises a defect in GCase activity. In some embodiments, the defect in GCase activity comprises a decreased enzymatic activity. In some embodiments, the disorder comprises alpha-synuclein dysregulation. In some embodiments, the disorder is a lysosomal storage disease, e.g., Gaucher disease, Fabry disease, Pompe disease, a mucopolysaccharidoses, or multiple system atrophy. Compositions described herein are especially suitable for treating Gaucher disease. In some embodiments, the disorder is a neurodegenerative disorder, e.g., Parkinson disease, Alzheimer's disease, or Lewy body dementia.

In another aspect is provided a method of treating a dysfunction in a GCase pathway comprising administering to a subject in need thereof any of the compositions described herein. In some embodiments, the subject is human.

In another aspect is provided a method of treating a dysfunction in a GCase pathway comprising administering to a subject a composition comprising from 0.5 to 5.0 mg/kg GCB and IFG, e.g., wherein IFG is in at least about a 1, 1.25, 1.5, 2, 2.5, 3, 4, or 5-fold molar excess to the GCB, wherein the composition is administered subcutaneously. In another aspect is provided a composition comprising from 0.5 to 5.0 mg/kg GCB and IFG, e.g., wherein the IFG is in at least about a 1, 1.25, 1.5, 2, 2.5, 3, 4, or 5-fold molar excess to the GCB, for use in a method of treating a dysfunction in a GCase pathway, wherein the composition is administered subcutaneously. In another aspect is provided the use of a composition comprising from 0.5 to 5.0 mg/kg GCB and IFG, e.g., wherein the IFG is in at least about a 1, 1.25, 1.5, 2, 2.5, 3, 4, or 5-fold molar excess to the GCB, in the manufacture of a medicament for a method of treating a dysfunction in a GCase pathway. In some embodiments, the IFG in the composition is administered in an amount which does not increase endogenous serum GCB activity. In some embodiments, the composition comprises from 0.8 to 4.0 mg/kg GCB. In some embodiments, the composition comprises from 1.0 to 3.0 mg/kg GCB. In some embodiments, the composition comprises from 1.2 to 2.0 mg/kg GCB. In some embodiments, the composition comprises about 1.5 mg/kg GCB. In some embodiments, the composition comprises 1.5 mg/kg GCB. In some embodiments, the composition comprises 2.0 to 5.0 mg/kg GCB. In some embodiments, the composition comprises 2.25 to 4.5 mg/kg GCB. In some embodiments, the composition comprises 2.25 to 3.75 mg/kg GCB. In some embodiments, the composition comprises 3.5 to 5.0 mg/kg GCB. In some embodiments, the IFG is in a 1 to 5 or a 1 to 10-fold molar ratio to the GCB. In some embodiments, the IFG is in a 2 to 10-fold molar ratio of GCB. In some embodiments, the IFG is in a 10 to 30-fold molar ratio to the GCB. In some embodiments, the IFG is in a 30 to 100-fold molar ratio to the GCB. In some embodiments, the IFG is in a 2.5 to 3.5-fold molar ratio to the GCB. In some embodiments, the IFG is in a 3-fold molar ratio to the GCB. In some embodiments, the exposure, activity, or bioavailability of the GCB is increased, e.g., relative to the exposure, activity, or bioavailability of an equivalent amount of GCB alone, administered IV. In some embodiments, the exposure, activity, or bioavailability of the GCB in the spleen is increased. In some embodiments, the exposure, activity, or bioavailability of the GCB in the liver is increased. In some embodiments, the exposure, activity, or bioavailability of the GCB in the serum is increased.

### BRIEF DESCRIPTION OF THE DRAWINGS

FIG. 1 is a flow diagram illustrating a process for preparing a glucocerebrosidase (GCB) and isofagomine (IFG) formulation.

FIGS. 2A and 2B illustrate SDS-PAGE testing of GCB samples on the first day after IFG was added (2A) and two weeks after IFG was added (2B). No pH adjustment of IFG was undertaken.

FIG. 3 shows Eppendorf tubes containing lyophilized solutions of pH-adjusted isofagomine tartrate (IFGT).

FIGS. 4A and 4B illustrate SDS-PAGE testing of GCB samples on the same day IFG was added (4A) and after three days of storage (4B). The IFG was pH adjusted.

FIG. 5 illustrates the results of a size exclusion chromatography (SEC) assay of pH-adjusted IFGT added to GCB.

FIGS. 6A and 6B illustrate the results of a size exclusion chromatography (SEC) assay of pH-adjusted IFG added to GCB.

FIGS. 7A-7D illustrate the results of surface plasmon resonance studies of IFG binding to GCB.

FIG. 8 illustrates the results from a nano-differential scanning fluorimetry (nano-DSF) assay evaluating GCB melting temperature changes with different IFG molar ratios ranging from 1:3 to 1:100 of GCB:IFG.

FIGS. 9A-9C illustrate the results of enzyme activity reactions performed on velaglucerase alpha preincubated with IFGT. FIG. 9A shows an inhibition curve with synthetic colorimetric pNP-GPS substrate. FIG. 9B shows an inhibition curve with synthetic fluorometric 4MU-GPS substrate. FIG. 9C shows inhibition with natural glycosphingolipid C12-GluCer substrate.

FIG. 10A shows the appearance of GCB/IFGT samples stored for three weeks at 40° C. FIG. 10B shows SDS-PAGE analysis of the GCB/IFGT samples stored at three weeks. The solutions of Groups 1-3 (G1, G2, G3) appear clear. The Group 4 (G4) solution appears cloudy.

FIG. 11 shows negative and positive controls for GCB immunohistochemical analysis (IHC) from a pharmacokinetic study of intravenous GCB and subcutaneous GCB with IFG in the cynomolgus monkey.

FIG. 12 shows staining of GCB in liver at 2× magnification at various time points after subcutaneous injection of GCB (upper panels) and intravenous injection of GCB (lower panels).

FIG. 13 shows staining of GCB in liver at 20× magnification at various time points after subcutaneous injection of GCB (upper panels) and intravenous injection of GCB (lower panels).

FIG. 14 shows staining of GCB in spleen at 2× magnification at various time points after subcutaneous injection of GCB (upper panels) and intravenous injection of GCB (lower panels).

FIG. 15 shows staining of GCB in spleen at 20× magnification at various time points after subcutaneous injection of GCB (upper panels) and intravenous injection of GCB (lower panels).

FIGS. 16A and 16B show the results of an assay of velaglucerase alfa protein and enzyme activity levels in liver and spleen homogenates after administration of velaglucerase alfa (16A) and velaglucerase alfa with IFGT in a 1:3 molar ratio (16B).

FIG. 16C shows the results of an assay of serum activity levels of GCB in cynomolgus monkeys after subcutaneous administration of velaglucerase alfa with IFGT.

FIGS. 17A and 17B show the results of an ECL ELISA assay of serum bioavailability of GCB (17A) and a GCB activity assay (17B) after subcutaneous administration of 4 mg/kg velaglucerase alfa and IFG at different molar ratios ranging from (1:3 to 1:100).

FIGS. 18A and 18B show the results of an ECL ELISA assay of the GCB content profile in the liver (18A) and spleen (18B) after intravenous administration of 10 mg/kg velaglucerase alfa or subcutaneous administration of 4 mg/kg velaglucerase alfa and IFG in a 1:100 molar ratio.

FIGS. 19A and 19B show the results of an ECL ELISA assay of GCB content in the liver (19A) and spleen (19B) after subcutaneous administration of 4 mg/kg velaglucerase alfa and IFG in a 1:3 molar ratio.

FIGS. 20A and 20B show the results of an ECL ELISA assay of serum bioavailability of GCB (20A) and a GCB activity assay (20B) after subcutaneous administration of 1.5 mg/kg velaglucerase alfa and IFG at different molar ratios ranging from (1:1 to 1:30).

FIG. 21 shows the results of an activity assay of VPRIV in human serum incubated at 37° C. with no IFG, 3 nM IFG, 10 nM IFG, 30 nM IFG, 100 nM IFG, 300 nM IFG and 1000 nM IFG.

### DETAILED DESCRIPTION

### Overview

Compositions comprising glucocerebrosidase (GCB) may benefit from increased stability, such as when the compositions are liquids. The three exposed free thiol groups in GCB can undergo reactions which lead to reduction in stability, e.g., by aggregation of GCB molecules. For example, in buffer at a pH of 6, typically 1-2% of the protein has aggregated upon one month of storage and about 15% has aggregated after 6 months of storage. While not wishing to be bound strictly by theory or mechanism, protein stability is influenced by a number of factors.

Adding isofagomine (IFG), e.g., isofagomine tartrate (IFGT), improves the stability of GCB in vitro, particularly when the IFG, e.g., IFGT, are adjusted to a pH of 6.0 before being

added to the GCB. IFG has the following structure:

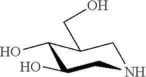

Without wishing to be bound by theory, IFG may interact with amino acid resides near the active site to lock GCB into a conformation that provides enhanced stability. See Shen, J. S. et al., Biochem. Biophys. Res. Comm., 2008, 369:1071-1075. IFG may also prevent GCB from aggregating because IFG can associate with GCB to render the GCB more compact and thermally more stable.

The present inventors have shown that the molar ratio of IFG to GCB is critical for stabilizing GCB in liquid compositions. As described in more detail throughout this application, compositions with a molar ratio of at least 1:2.5 (GCB:IFG) (i.e. 1:x (wherein x is at least 2.5)) may have substantially less GCB aggregation and degradation. There may be substantially more aggregation and degradation of GCB with molar ratios substantially below 1:2.5.

The present inventors have also shown that compositions with a molar ratio of IFG/IFGT to GCB of at least 1:2.5 (GCB:IFG) have improved GCB bioavailability, activity, tissue exposure, and systemic exposure when administered subcutaneously. The improved bioavailability may be detected by one or more of increased tissue staining of GCB in liver, increased tissue staining of GCB in spleen, an increased concentration of GCB in serum, and an increased GCB activity in serum. Improved systemic exposure may be assayed by measuring the protein concentration of GCB or the enzyme activity of GCB in serum. Adding IFG, e.g., IFGT, to GCB, in a molar ratio of at least 1:2.5 (GCB:IFG) can allow for the bioavailability, activity, tissue exposure, or systemic exposure of GCB in a subcutaneous formulation to be similar to, or greater than, GCB bioavailability, activity, tissue exposure, or systemic exposure in an intravenous formulation, particularly a formulation without IFG.

### Definitions

The term “subject” refers to any mammal, including but not limited to, any animal classified as such, including humans, non-human primates, primates, baboons, chimpanzees, monkeys, rodents (e.g., mice, rats), rabbits, cats, dogs, horses, cows, sheep, goats, pigs, etc. The term “subject” can be used interchangeably with the term “patient.”

The term “isolated” refers to a molecule that is substantially free of its natural environment. For instance, an isolated protein is substantially free of cellular material or other proteins from the cell or tissue source from which it is derived. Preparations comprising isolated protein are sufficiently pure to be administered as a therapeutic composition, or at least 70% to 80% (w/w) pure, more preferably, at least 80% to 90% (w/w) pure, even more preferably, 90 to 95% pure; and, most preferably, at least 95%, 96%, 97%, 98%, 99%, 99.5%, 99.8% or 100% (w/w) pure.

As used herein, the term “about” refers to up to +1-10% of the value qualified by this term. For example, about 50 mM refers to 50 mM+/−5 mM; about 4% refers to 4%+/−0.4%.

The phrases “parenteral administration”, “administered parenterally” and “administer parenterally” as used herein refer to modes of administration other than enteral and topical administration, usually by injection, and include, without limitation, intravenous (IV), intramuscular, intraarterial, intrathecal, intracapsular, intraorbital, intracardiac, intradermal, intraperitoneal, transtracheal, subcutaneous (SC), subcuticular, intraarticular, subcapsular, subarachnoid, intraspinal, epidural, and intrasternal injection and infusion.

The terms “therapeutically effective dose,” and “therapeutically effective amount,” refer to that amount of a compound that results in prevention of symptoms, for example, prevention of 80%, 85%, 90%, 95%, 96%, 97%, 98%, or 99% of symptoms, e.g., symptoms of Gaucher disease in a subject diagnosed as having Gaucher disease), delay of onset of symptoms, or amelioration of symptoms of Gaucher disease. A therapeutically effective amount will, for example, be sufficient to treat, prevent, reduce the severity, delay the onset, and/or reduce the risk of occurrence of one or more symptoms of a disorder associated with Gaucher disease. The effective amount can be determined by methods well known in the art and as described in subsequent sections of this description.

The terms “treatment” and “therapeutic method” refer to treatment of an existing disorder and/or prophylactic/preventative measures. Those in need of treatment may include individuals already having a particular medical disorder, as well as those at risk of having, or who may ultimately acquire the disorder. The need for treatment is assessed, for example, by the presence of one or more risk factors associated with the development of a disorder, the presence or progression of a disorder, or likely receptiveness to treatment of a subject having the disorder. Treatment may include slowing or reversing the progression of a disorder.

The term “treating” refers to administering a therapy in an amount, manner, and/or mode effective to improve or prevent a condition, symptom, or parameter associated with a disorder (e.g., a disorder described herein) or to prevent onset, progression, or exacerbation of the disorder, to either a statistically significant degree or to a degree detectable to one skilled in the art. Accordingly, treating can achieve therapeutic and/or prophylactic benefits. An effective amount, manner, or mode can vary depending on the subject and may be tailored to the subject. In certain embodiments, treatment of a disorder related to a dysfunction in a GCase pathway (e.g., Gaucher disease), is a treatment which results in one or more of an increase in hemoglobin concentration, an increase in platelet level, a decrease in liver volume, a decrease in spleen volume, or a change in a skeletal parameter (e.g., an increase in bone mineral density), e.g., in a subject who has not been treated for the dysfunction in a GCase pathway. In certain embodiments, treatment of a disorder related to a dysfunction in a GCase pathway (e.g., Gaucher disease), is a treatment which results in one or more of an increase in hemoglobin concentration, an increase in platelet level, a decrease in liver volume, a decrease in spleen volume, or a change in a skeletal parameter (e.g., an increase in bone mineral density), or maintenance of one or more of these parameters, e.g., in a subject who has been treated for the dysfunction in a GCase pathway.

The term “combination” refers to the use of the two or more agents or therapies to treat the same patient, wherein the use or action of the agents or therapies overlap in time. The agents or therapies can be administered at the same time (e.g., as a single formulation that is administered to a patient or as two separate formulations administered concurrently) or sequentially in any order.

The terms “sustained release”, “sustained release delivery” and “sustained release drug delivery” as used herein mean that a single administration of drug maintains the effective concentration of the drug in blood for a long period, for example, 12 hours or longer. For example, the general administration route of polypeptides is subcutaneous, intramuscular or intravenous (IV) injection.

The term “salts” embraces addition salts of free acids or free bases. The term “pharmaceutically-acceptable salt” refers to salts which possess toxicity profiles within a range that affords utility in pharmaceutical applications. Salts that are not pharmaceutically acceptable salts may still be useful in synthesis, purification or formulation on account of properties such as high crystallinity.

The term “unit” with respect to GCB, velaglucerase, or velaglucerase alfa refers to the amount of these that is required to convert one micromole of p-nitrophenyl beta-D-glucopyranoside to p-nitrophenol, or 4-methylumbelliferone beta-D-glucopyranoside to 4-methylumbelliferone, per minute at 37° C.

### Glucocerebrosidase

Velaglucerase is human β-glucocerebrosidase produced by gene-activation in a human cell line, such as by targeted recombination with a promoter that activates the endogenous β-glucocerebrosidase gene in the selected human cell line. Velaglucerase is secreted as a monomeric glycoprotein of approximately 63 kDa. Velaglucerase is composed of 497 amino acids with a sequence identical to the natural human protein. See Zimran et al., Blood Cells Mol. Dis., 2007, 39: 115-118.

The glycosylation of velaglucerase alfa may be altered by using kifunensine, a mannosidase I inhibitor, during cell culture so as to produce a secreted protein containing primarily high-mannose type glycans having 6-9 mannose units per glycan, as described in more detail in WO 2013/130963.

Imiglucerase (Cerezyme®) is another form of recombinant human β-glucocerebrosidase. Imiglucerase is recombinantly produced in Chinese Hamster Ovary (CHO) cells.

Taliglucerase alfa (Elelyso® or Uplyso®) is a recombinant glucocerebrosidase (prGCB) expressed in plant cells. Plant recombinant glucocerebrosidase can be obtained by methods described at least in U.S. Patent Publication Nos. 2009/0208477 and 2008/0038232 and PCT Publication Nos. WO 2004/096978 and WO 2008/132743.

Any of the recombinant GCB can be produced using bioreactors and production scale synthesis methods known in the art. Any number of production scale purification systems can be used.

### Isofagomine

Various alternative forms of isofagomine can be used. These include any of isofagomine tartrate, isofagomine HCl, isofagomine free base and isofagomine citrate. In some embodiments, isofagomine comprises one or more of isofagomine HCl, isofagomine free base and isofagomine citrate. In some embodiments, isofagomine comprises isofagomine tartrate.

Isofagomine HCl is described in U.S. Pat. Nos. 5,844,102 and 7,501,439. Isofagomine HCl is a yellow colored solid with a low melting point. Isofagomine free base can be prepared by converting isofagomine HCl to the free base form.

In any of the aspects and embodiments described herein, isofagomine may not be in the form of isofagomine tartrate, or the GCB/IFG composition may not comprise isofagomine tartrate.

### Isofagomine Tartrate

Isofagomine tartrate (IFGT) is a specific form of isofagomine (IFG) that may be used in the various embodiments disclosed herein, and is especially suitable for practicing the invention. IFGT has the following formula:

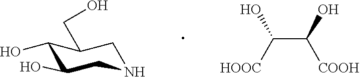

IFGT has improved characteristics as compared to IFG, which include improved synthetic manufacturability. For example, it may be easier to purify IFGT in solvents such as water and ethanol. IFGT has greater stability than other known salt forms of isofagomine. IFGT is also particularly suitable for industrial scale production, e.g., production of greater than 1 kg of product.

A composition comprising GCB and IFG is sometimes referred to throughout this application as a GCB/IFG composition. A composition comprising GCB and IFGT is sometimes referred to throughout this application as a GCB/IFGT composition.

### Molar Ratios of GCB to IFG/IFGT

In various embodiments, the composition comprises a glucocerebrosidase (GCB) and an isofagomine (IFG), e.g., isofagomine tartrate (IFGT), in a molar ratio of at least about 1:1, 1:1.5, 1:2, or 1:2.5 (GCB:IFG). The molar ratio of GCB to IFG, e.g., GCB to IFGT, can be 1:1, 1:1.5, 1:2, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, 1:4.0, 1:4.1, 1:4.2, 1:4.3, 1:4.4, 1:4.5, 1:4.6, 1:4.7, 1:4.8, 1:4.9, 1:5.0, 1:5.1, 1:5.2, 1:5.3, 1:5.4, 1:5.5, 1:5.6, 1:5.7, 1:5.8, 1:5.9, 1:6.0, 1:6.1, 1:6.2, 1:6.3, 1:6.4, 1:6.5, 1:6.6, 1:6.7, 1:6.8, 1:6.9, 1:7.0, 1:7.1, 1:7.2, 1:7.3, 1:7.4, 1:7.5, 1:7.6, 1:7.7, 1:7.8, 1:7.9, 1:8.0, 1:8.1, 1:8.2, 1:8.3, 1:8.4, 1:8.5, 1:8.6, 1:8.7, 1:8.8, 1:8.9, 1:9.0, 1:9.1, 1:9.2, 1:9.3, 1:9.4, 1:9.5, 1:9.6, 1:9.7, 1:9.8, 1:9.9, 1:10.0, 1:10.1, 1:10.2, 1:10.3, 1:10.4, 1:10.5, 1:10.6, 1:10.7, 1:10.8, 1:10.9, 1:11.0, 1:11.1, 1:11.2, 1:11.3, 1:11.4, 1:11.5, 1:11.6, 1:11.7, 1:11.8, 1:11.9, 1:12.0, 1:12.1, 1:12.2, 1:12.3, 1:12.4, 1:12.5, 1:12.6, 1:12.7, 1:12.8, 1:12.9, 1:13.0, 1:13.1, 1:13.2, 1:13.3, 1:13.4, 1:13.5, 1:13.6, 1:13.7, 1:13.8, 1:13.9, 1:14.0, 1:14.1, 1:14.2, 1:14.3, 1:14.4, 1:14.5, 1:14.6, 1:14.7, 1:14.8, 1:14.9, 1:15.0, 1:15.1, 1:15.2, 1:15.3, 1:15.4, 1:15.5, 1:15.6, 1:15.7, 1:15.8, 1:15.9, 1:16.0, 1:16.1, 1:16.2, 1:16.3, 1:16.4, 1:16.5, 1:16.6, 1:16.7, 1:16.8, 1:16.9, 1:17.0, 1:17.1, 1:17.2, 1:17.3, 1:17.4, 1:17.5, 1:17.6, 1:17.7, 1:17.8, 1:17.9, 1:18.0, 1:18.1, 1:18.2, 1:18.3, 1:18.4, 1:18.5, 1:18.6, 1:18.7, 1:18.8, 1:18.9, 1:19.0, 1:19.1, 1:19.2, 1:19.3, 1:19.4, 1:19.5, 1:19.6, 1:19.7, 1:19.8, 1:19.9, 1:20.0, 1:20.1, 1:20.2, 1:20.3, 1:20.4, 1:20.5, 1:20.6, 1:20.7, 1:20.8, 1:20.9, 1:21.0, 1:21.1, 1:21.2, 1:21.3, 1:21.4, 1:21.5, 1:21.6, 1:21.7, 1:21.8, 1:21.9, 1:22.0, 1:22.1, 1:22.2, 1:22.3, 1:22.4, 1:22.5, 1:22.6, 1:22.7, 1:22.8, 1:22.9, 1:23.0, 1:23.1, 1:23.2, 1:23.3, 1:23.4, 1:23.5, 1:23.6, 1:23.7, 1:23.8, 1:23.9, 1:23.9, 1:24.0, 1:24.1, 1:24.2, 1:24.3, 1:24.4, 1:24.5, 1:24.6, 1:24.7, 1:24.8, 1:24.9, 1:25.0, 1:25.1, 1:25.2, 1:25.3, 1:25.4, 1:25.5, 1:25.6, 1:25.7, 1:25.8, 1:25.9, 1:26.0, 1:26.1, 1:26.2, 1:26.3, 1:26.4, 1:26.5, 1:26.6, 1:26.7, 1:26.8, 1:26.9, 1:27.0, 1:27.1, 1:27.2, 1:27.3, 1:27.4, 1:27.5, 1:27.6, 1:27.7, 1:27.8, 1:27.9, 1:28.0, 1:28.1, 1:28.2, 1:28.3, 1:28.4, 1:28.5, 1:28.6, 1:28.7, 1:28.8, 1:28.9, 1:29.0, 1:29.1, 1:29.2, 1:29.3, 1:29.4, 1:29.5, 1:29.6, 1:29.7, 1:29.8, 1:29.9, or 1:30.0.

The molar ratio of GCB to IFG, e.g., GCB to IFGT, can be from 1:2.5 to 1:3.5, from 1:2.6 to 1:3.4, from 1:2.7 to 1:3.5, from 1:2.7 to 1:3.4, from 1:2.5 to 1:3.3, from 1:2.8 to 1:3.5, from 1:2.8 to 1:3.3, from 1:2.7 to 1:3.2, from 1:2.6 to 1:3.1, from 1:2.5 to 1:3.0, from 1:2.9 to 1:3.3, from 1:2.8 to 1:3.2, from 1:2.7 to 1:3.1, from 1:2.6 to 1:3.0, from 1:2.5 to 1:2.9, from 1:3.0 to 1:3.4, or from 1:3.1 to 1:3.5.

The molar ratio of GCB to IFG, e.g., GCB to IFGT, can be from 1:7 to 1:33, from 1:8 to 1:32, from 1:9 to 1:33, from 1:7 to 1:31, from 1:9 to 1:31, from 1:8 to 1:30, from 1:7 to 1:29, from 1:10 to 1:32, from 1:11 to 1:33, from 1:7 to 1:29, from 1:10 to 1:30, from 1:9 to 1:29, from 1:8 to 1:28, from 1:7 to 1:27, from 1:11 to 1:31, from 1:12 to 1:32, from 1:13 to 1:33, from 1:11 to 1:29, from 1:10 to 1:28, from 1:9 to 1:27, from 1:8 to 1:26, from 1:7 to 1:25, from 1:12 to 1:30, from 1:13 to 1:31, from 1:14 to 1:32, from 1:15 to 1:33, from 1:13 to 1:29, from 1:12 to 1:28, from 1:11 to 1:27, from 1:10 to 1:26, from 1:9 to 1:25, from 1:8 to 1:24, from 1:7 to 1:23, from 1:14 to 1:30, from 1:15 to 1:31, from 1:16 to 1:32, from 1:17 to 1:33, from 1:14 to 1:28, from 1:13 to 1:27, from 1:12 to 1:26, from 1:11 to 1:25, from 1:10 to 1:24, from 1:9 to 1:23, from 1:8 to 1:22, from 1:7 to 1:21, from 1:15 to 1:29, from 1:16 to 1:30, from 1:17 to 1:31, from 1:18 to 1:32, from 1:19 to 1:33, from 1:15 to 1:27, from 1:14 to 1:26, from 1:13 to 1:25, from 1:12 to 1:24, from 1:11 to 1:23, from 1:10 to 1:22, from 1:9 to 1:21, from 1:8 to 1:20, from 1:7 to 1:19, from 1:16 to 1:28, from 1:17 to 1:29, from 1:18 to 1:30, from 1:19 to 1:31, from 1:20 to 1:32, or from 1:21 to 1:33.

The molar ratio of GCB to IFG, e.g., GCB to IFGT, can be from 1:16 to 1:26, from 1:15 to 1:25, from 1:14 to 1:24, from 1:13 to 1:23, from 1:12 to 1:22, from 1:11 to 1:31, from 1:10 to 1:30, from 1:9 to 1:29, from 1:8 to 1:28, from 1:7 to 1:27, from 1:17 to 1:27, from 1:18 to 1:28, from 1:19 to 1:29, from 1:20 to 1:30, from 1:21 to 1:31, from 1:22 to 1:32, from 1:23 to 1:33, from 1:17 to 1:25, from 1:14 to 1:24, from 1:13 to 1:23, from 1:12 to 1:22, from 1:11 to 1:21, from 1:10 to 1:20, from 1:9 to 1:19, from 1:18 to 1:26, from 1:19 to 1:27, from 1:20 to 1:28, from 1:21 to 1:29, from 1:22 to 1:30, from 1:23 to 1:31, from 1:18 to 1:24, from 1:17 to 1:23, from 1:16 to 1:22, from 1:15 to 1:21, from 1:14 to 1:20, from 1:13 to 1:19, from 1:12 to 1:18, from 1:11 to 1:17, from 1:19 to 1:25, from 1:20 to 1:26, from 1:21 to 1:27, from 1:22 to 1:28, from 1:23 to 1:29, from 1:24 to 1:30, from 1:19 to 1:23, from 1:17 to 1:21, from 1:15 to 1:19, from 1:13 to 1:17, from 1:11 to 1:15, from 1:9 to 1:13, from 1:7 to 1:11, from 1:21 to 1:25, from 1:23 to 1:27, from 1:25 to 1:29, from 1:27 to 1:31, from 1:29 to 1:33, from 1:20 to 1:23, from 1:18 to 1:21, from 1:16 to 1:19, from 1:14 to 1:17, from 1:12 to 1:15, from 1:10 to 1:13, from 1:8 to 1:11, from 1:22 to 1:25, from 1:24 to 1:27, from 1:26 to 1:29, from 1:28 to 1:31, or from 1:30 to 1:33.

The molar ratio of GCB to IFG, e.g., GCB to IFGT, can be 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:35, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, or 1:100.

The molar ratio of GCB to IFG, e.g., GCB to IFGT, can be from 1:30 to 1:100, from 1:30 to 1:80, from 1:40 to 1:90, from 1:50 to 1:100, from 1:30 to 1:60, from 1:40 to 1:70, from 1:50 to 1:80, from 1:60 to 1:90, from 1:70 to 1:100, from 1:30 to 1:50, from 1:40 to 1:60, from 1:50 to 1:70, from 1:60 to 1:80, from 1:70 to 1:90, from 1:80 to 1:100, from 1:30 to 1:40, from 1:40 to 1:50, from 1:50 to 1:60, from 1:60 to 1:70, from 1:70 to 1:80, from 1:80 to 1:90, or from 1:90 to 1:100.

In other various embodiments described herein, the composition comprises a glucocerebrosidase (GCB) and an isofagomine tartrate (IFGT) in a molar ratio of at least about 1:2.5.

In other various embodiments described herein, the composition comprises a glucocerebrosidase (GCB) and an isofagomine citrate in a molar ratio of at least about 1:2.5.

In other various embodiments described herein, the composition comprises a glucocerebrosidase (GCB) and an isofagomine HCl in a molar ratio of at least about 1:2.5.

In other various embodiments described herein, the composition comprises a glucocerebrosidase (GCB) and an isofagomine free base in a molar ratio of at least about 1:2.5.

In other various embodiments described herein, the composition comprises a glucocerebrosidase (GCB) and an isofagomine that does not comprise IFGT in a molar ratio of at least about 1:2.5.

### GCB Concentration

The concentration of GCB in any of the compositions can be from about 0.1 to about 40 mg/ml, from about 0.5 to about 10 mg/ml, from about 5 to about 15 mg/ml, from about 10 to about 20 mg/ml, from about 15 to about 25 mg/ml, from about 20 to about 30 mg/ml, from about 25 to about 35 mg/ml, from about 30 to about 40 mg/ml, from about 2 to about 8 mg/ml, from about 5 to about 11 mg/ml, from about 8 to about 14 mg/ml, from about 11 to about 17 mg/ml, from about 14 to about 20 mg/ml, from about 17 to about 23 mg/ml, from about 20 to about 26 mg/ml, from about 23 to about 29 mg/ml, from about 26 to about 32 mg/ml, from about 29 to about 35 mg/ml, from about 32 to about 38 mg/ml, from about 2 to about 5 mg/ml, from about 5 to about 8 mg/ml, from about 8 to about 11 mg/ml, from about 11 to about 14 mg/ml, from about 14 to about 17 mg/ml, from about 17 to about 20 mg/ml, from about 20 to about 23 mg/ml, from about 23 to about 26 mg/ml, from about 26 to about 29 mg/ml, from about 29 to about 32 mg/ml, from about 32 to about 35 mg/ml, from about 35 to about 38 mg/ml, about 0.5 mg/ml, about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, or about 40 mg/ml.

The concentration of GCB can be from 50 Units/ml to 200 Units/ml, 70 Units/ml to 160 Units/ml, 80 Units/ml to 175 Units/ml, 90 Units/ml to 190 Units/ml, 60 Units/ml to 145 Units/ml, 50 Units/ml to 130 Units/ml, 80 Units/ml to 140 Units/ml, 70 Units/ml to 120 Units/ml, 60 Units/ml to 100 Units/ml, 50 Units/ml to 85 Units/ml, 90 Units/ml to 160 Units/ml, 100 Units/ml to 180 Units/ml, 120 Units/ml to 200 Units/ml, 90 Units/ml to 125 Units/ml, 80 Units/ml to 105 Units/ml, 70 Units/ml to 100 Units/ml, 60 Units/ml to 90 Units/ml, 50 Units/ml to 80 Units/ml, 100 Units/ml to 140 Units/ml, 115 Units/ml to 160 Units/ml, 130 Units/ml to 180 Units/ml, 145 Units/ml to 200 Units/ml, 100 Units/ml to 115 Units/ml, 90 Units/ml to 105 Units/ml, 80 Units/ml to 95 Units/ml, 70 Units/ml to 85 Units/ml, 60 Units/ml to 75 Units/ml, 50 Units/ml to 65 Units/ml, 110 Units/ml to 125 Units/ml, 120 Units/ml to 135 Units/ml, 130 Units/ml to 145 Units/ml, 140 Units/ml to 160 Units/ml, 160 Units/ml to 180 Units/ml, 180 Units/ml to 200 Units/ml, about 50 Units/ml, about 60 Units/ml, about 70 Units/ml, about 80 Units/ml, about 90 Units/ml, about 100 Units/ml, about 110 Units/ml, about 120 Units/ml, about 130 Units/ml, about 140 Units/ml, about 150 Units/ml, about 160 Units/ml, about 170 Units/ml, about 180 Units/ml, about 190 Units/ml, about 200 Units/ml, 50 Units/ml, 60 Units/ml, 70 Units/ml, 80 Units/ml, 90 Units/ml, 100 Units/ml, 110 Units/ml, 120 Units/ml, 130 Units/ml, 140 Units/ml, 150 Units/ml, 160 Units/ml, 170 Units/ml, 180 Units/ml, 190 Units/ml, or 200 Units/ml.

### Pharmaceutical Compositions

A pharmaceutical composition may include a “therapeutically effective amount” of a GCB/IFG, e.g., GCB/IFGT, composition described herein. Such effective amounts can be determined based on the effect of the administered composition. A therapeutically effective amount of a GCB/IFG, e.g., GCB/IFGT, composition may also vary according to factors such as the disease state, age, sex, and weight of the individual, and the ability of the composition to elicit a desired response in the individual, e.g., amelioration of at least one symptom of a condition or disorder, e.g., a glucocerebrosidase deficiency, e.g., Gaucher disease. A therapeutically effective amount is also one in which any toxic or detrimental effects of the composition are outweighed by the therapeutically beneficial effects.

The GCB/IFG composition may be free of IFGT.

A pharmaceutical composition of the invention can be formulated to be compatible with its intended route of administration. For example, a GCB/IFG, e.g., GCB/IFGT, composition can be administered by a parenteral mode, e.g., intravenous, subcutaneous, intraperitoneal, or intramuscular injection. In various embodiments, the route of administration is intravenous. In various embodiments, the route of administration is subcutaneous. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH of pharmaceutical compositions can be adjusted with acids or bases, such as hydrochloric acid or sodium hydroxide. The parenteral preparation can be enclosed in ampoules, disposable syringes or multiple dose vials made of glass or plastic.

pH

pH can have an influence on the stability of GCB in the various GCB/IFG and GCB/IFGT compositions described herein. pH can affect the conformation and/or aggregation and/or degradation and/or the reactivity of the GCB. For example, at a higher pH, oxygen can be more reactive. The pH is preferably less than 7.0, more preferably in the range of about 4.5 to about 6.5, more preferably about 5.0 to about 6.0, and more preferably about 5.5 to about 5.8, more preferably about 5.7. With GCB, aggregation can reach undesirable levels at a pH above 7.0 and degradation (e.g., fragmentation) can reach undesirable levels at a pH under 4.5 or 5.0, or at a pH above 6.5 or 7.0.

A candidate pH can be tested for by providing a test GCB/IFG, e.g., GCB/IFGT, composition, adjusting the composition to a candidate pH, and purging the composition of oxygen. The stability of the GCB in the composition at the candidate pH may be measured, e.g., as a percent aggregation or degradation, at a predetermined time. The measured stability may be compared with one or more standards. For example, a suitable standard would be a composition similar to the test compositions except that the pH of the composition is not adjusted. The stabilities of the pH-adjusted and non pH-adjusted compositions may then compared. A GCB/IFG, e.g., GCB/IFGT, composition may be more suitable if the GCB is more stable than that of a comparative standard composition. Suitability can be shown by the test treatment increasing stability as compared with this standard. For example, if the comparative standard GCB/IFG composition has a pH of 5.5 but increased GCB stability is seen when the GCB/IFG composition has a pH of 6.3, then the composition at pH of 6.3 is more suitable because GCB is more stable at pH 6.3 than at pH 5.5.

Buffers that can be used to adjust the pH of a protein composition include histidine, citrate, phosphate, glycine, succinate, acetate, glutamate, Tris, tartrate, aspartate, maleate, and lactate.

### GCB Stability Assays

Protein stability can be measured by measuring protein aggregation or protein degradation. Protein aggregation can be determined by various methods that include, for example, size exclusion chromatography (SEC), non-denaturing PAGE, or other methods for determining size, etc. Protein degradation can be determined, for example, by reverse phase HPLC, non-denaturing PAGE, ion-exchange chromatography, peptide mapping, or similar methods.

Stability, as used herein, includes parameters such as protein structure (e.g., minimizing or preventing changes in protein structure, e.g., protein aggregation or protein degradation (e.g., fragmentation)) and/or a biological activity of the protein, e.g., the ability to convert substrate into product.

GCB stability can be measured, e.g., by measuring protein aggregation, protein degradation, or levels of a biological activity of the GCB. Aggregation of GCB can be determined, by various methods including size exclusion chromatography, non-denaturing PAGE, and other methods for determining size. For example, the composition can have less than a 1, 5, 10, 15, 20, 25, 30, 35, 40, 45, or 50% increase in the amount of GCB protein aggregation (e.g., as measured by size exclusion chromatography) as compared to the amount of protein aggregation that was in the composition prior to storage (e.g., storage at a temperature of 2-8° C. for a period of up to 3, 6, 9, 12, or 24 months (or longer)).

Protein degradation can be determined by various methods including reverse phase HPLC, non-denaturing PAGE, ion-exchange chromatography, peptide mapping, or similar methods. As an example, the composition can have less than a 1, 5, 10, 15, 20, 25, 30, 35, 40, 45, or 50% increase in the amount of GCB degradation (e.g., as measured by reverse phase HPLC) as compared to the amount of GCB degradation that was in the composition prior to storage (e.g., storage at a temperature of 2-8° C. for a period of up to 3, 6, 9, 12, or 24 months (or longer)). The biological activity of GCB can be measured, e.g., by in vitro or in vivo assays, e.g., ELISA (e.g., to measure binding or enzymatic activity) and other enzymatic assays (e.g., spectrophotometric, fluorimetric, calorimetric, chemiluminescent, radiometric, or chromatographic assays), kinase assays, and so forth. As an example, the composition can have less than a 1, 5, 10, 15, 20, 25, 30, 35, 40, 45, or 50% decrease in a biological activity of GCB (e.g., enzymatic activity, e.g., as measured by an in vitro assay) as compared to the amount of the biological activity that was in the composition prior to storage (e.g., storage at a temperature of 2-8° C. for a period of up to 3, 6, 9, 12, or 24 months (or longer)).

### Antioxidants and Stabilizers

The GCB/IFG and GCB/IFGT compositions described herein may further comprise an antioxidant. One suitable antioxidant is cysteine. Cysteine may be present at from 0.030% to 0.100%, 0.050% to 0.080%, 0.040% to 0.070%, 0.030% to 0.060%, 0.060% to 0.090%, 0.070% to 0.100%, 0.065% to 0.080%, 0.060% to 0.075%, 0.055% to 0.070%, 0.050% to 0.065%, 0.070% to 0.085%, 0.075% to 0.090%, about 0.065%, about 0.070%, about 0.075%, about 0.080%, 0.065%, 0.070%, 0.075%, or 0.080%. Without wishing to be bound by theory, cysteine may further stabilize GCB.

The GCB/IFG and GCB/IFGT compositions described herein may further comprise a carbohydrate such as sucrose or trehalose. The carbohydrate, e.g., sucrose or trehalose, may be present at from 12% to 19%, 13% to 18%, 14% to 17%, 12% to 15%, 13% to 16%, 15% to 17%, about 16%, or 16%. Without wishing to be bound by theory, sucrose or trehalose may further stabilize GCB by decreasing the availability of thiol (—SH) groups.

The GCB/IFG and GCB/IFGT compositions herein may further comprise a detergent. The detergent may be polysorbate 20 (which is especially suitable for practicing the invention) or any number of poloxomer-based compounds.

In certain embodiments, the stability of GCB is at least 5-80% greater (e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% greater), under pre-selected conditions, than the stability of GCB in a composition which differs by lacking the carbohydrate (sucrose or trehalose), the antioxidant, or both the carbohydrate and the antioxidant.

The GCB/IFG and GCB/IFGT compositions may be purged of oxygen prior to storage in a container. Further, the container is ideally gas tight so as to prevent intrusion of oxygen. The GCB in the compositions described herein, e.g., liquid compositions containing GCB, may have prolonged stability. For example, under pre-selected conditions, e.g., upon storage in a gas tight container, at a temperature of 2-8° C. for a period of up to 3, 6, 9, 12, or 24 months (or in some embodiments longer), GCB in the composition will retain at least 50, 55, 60, 65, 70, 75, 80, 85, 90, 95, 99, or 100% of the stability it had prior to storage.

A suitable protein concentration can be tested for by providing a composition containing 0.075% cysteine, 16% sucrose, adjusting the pH to 5.7, adjusting the GCB to a candidate concentration, and purging the composition of 02. The stability of GCB in the GCB/IFG, e.g., GCB/IFGT, composition at the candidate concentration, measured, e.g., as a percent aggregation or degradation, at a predetermined time is compared with one or more standards. The stabilities of the GCB at each concentration are compared. Suitability can be shown by the candidate concentration having comparable or better effects on stability than a concentration described herein.

GCB stability can be measured by any of the methods described throughout this application, e.g., by measuring protein aggregation or protein degradation. Protein aggregation can be determined, e.g., by size exclusion chromatography, non-denaturing PAGE, or other methods for determining size, etc. Protein degradation can be determined, e.g., by reverse phase HPLC, non-denaturing PAGE, ion-exchange chromatography, SEC, SEC HPLC, peptide mapping, or similar methods.

### Surfactants

The GCB/IFG and GCB/IFGT compositions described herein may further comprise one or more surfactants. Without wishing to be bound by theory, surfactants can increase protein stability, such as by providing an air/liquid interface that can reduce protein degradation upon shaking or during shipment. A surfactant may be selected that increases protein stability, such as by not causing protein degradation, in a particular liquid composition. An exemplary surfactant is poloxamer 188 or Pluronic F68. The surfactant can be present in an amount between about 0.005% and about 5%, e.g., between about 0.01% and about 1%, e.g., about 0.025% and about 0.5%, e.g., about 0.03% and about 0.25%, e.g., about 0.04 to about 0.1%, e.g., about 0.05% to about 0.075%, e.g., 0.05%. An ideal surfactant is one that is not modified or cleaved by GCB.

For example, a candidate surfactant can be tested by providing a composition containing 2 mg/ml GCB, an amount of IFG, 0.075% cysteine, 16% sucrose, then adjusting the pH to 5.7, then adding the candidate surfactant, and purging the composition of O₂. The stability of the GCB/IFG composition containing the candidate surfactant is measured, e.g., as a percent aggregation or degradation, at a predetermined time compared with one or more standards. For example, a suitable standard would be a composition similar to the test conditions except that a surfactant is not added to the composition. The stabilities of the treated (containing the surfactant) and untreated (lacking a surfactant) compositions may be compared in conditions simulating “real world” scenarios, e.g., storage and shipping. A standard can be a composition similar to the test composition except that another surfactant is used instead of poloxamer 188. Poloxamer 188 would then be a standard for the basis of comparison. Suitability can be shown by the candidate surfactant having comparable or better effects on stability than a surfactant described herein. If the candidate surfactant is determined to be suitable (e.g., it increases stability of the composition as compared to one of the standards), the concentration of the candidate surfactant can be refined. For example, the concentration can be increased or decreased over a range of values and compared to the standard and to the other concentrations being tested to determine which concentration causes the greatest increase in stability.

Alternatively, a combination of two or more surfactants is used in the compositions described herein. The suitability of the combination can be tested as described above by comparing the stability of a GCB/IFG composition with the test combination of surfactants with the stability of a GCB/IFG composition with poloxamer 188.

Protein stability can be measured, e.g., by measuring protein aggregation or protein degradation. Protein aggregation can be determined, e.g., by size exclusion chromatography, non-denaturing PAGE, or other methods for determining size, etc. Protein degradation can be determined, e.g., by reverse phase HPLC, non-denaturing PAGE, ion-exchange chromatography, peptide mapping, or similar methods.

### Pharmaceutically Acceptable Salt

The pharmaceutical composition may further comprise a salt or pharmaceutically acceptable salt.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, .beta.-hydroxybutyric, salicylic, galactaric, oxalic, malonic and galacturonic acid. Examples of pharmaceutically unacceptable acid addition salts include, for example, perchlorates and tetrafluoroborates. All of these acid addition salts may be prepared from isofagomine or GCB by reacting, for example, the appropriate acid with the compound.

Suitable pharmaceutically acceptable base addition salts of isofagomine include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. All of these base addition salts may be prepared from isofagomine by reacting, for example, the appropriate base with the compound.

### Pharmaceutical Carriers

The GCB-containing pharmaceutical compositions can include one or more pharmaceutically acceptable carriers. As used herein, the language “pharmaceutically acceptable carrier” is intended to include any and all solvents, excipients, dispersion media, coatings, antibacterial and antifungal agents, isotonic and adsorption delaying agents, and the like, compatible with pharmaceutical administration. Pharmaceutical formulation is a well-established art, and is further described, e.g., in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X). Except insofar as any conventional media or agent is incompatible with the active compound, such media can be used in the compositions of the invention. Supplementary active compounds can also be incorporated into the compositions.

The pharmaceutical compositions described herein may further include carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

For IV administration, suitable carriers include physiological saline, bacteriostatic water, CREMOPHOR EL™ (BASF, Parsippany, N.J.) or phosphate buffered saline (PBS). In all cases, the composition must be sterile and should be fluid to the extent that easy syringability exists. The composition should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Proper fluidity can be maintained, for example, by the use of a coating such as lecithin, by the maintenance of the required particle size in the case of dispersion, and by the use of surfactants. Prevention of microorganism action can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged stability of the injectable compositions can be brought about by including an agent which delays adsorption, for example, aluminum monostearate, human serum albumin and gelatin.

Sterile injectable solutions can be prepared by incorporating GCB/IFG in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the composition of sterile injectable solutions, the preferred methods of composition are vacuum drying and freeze-drying, e.g., lyophilization, which yields a powder of the active ingredient plus any additional desired ingredient from a previously sterile-filtered solution thereof.

The active compounds (e.g., GCB compositions described herein) can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

### Packaging and Delivery

The GCB/IFG and GCB/IFGT compositions described herein can be administered with various medical devices. For example, a composition described herein can be administered with a needle-less hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules also are known.

The GCB/IFG and GCB/IFGT compositions described herein can be packaged in a two chamber syringe. For example, the GCB/IFG and GCB/IFGT compositions in lyophilized form can be placed into a first syringe chamber and a liquid can be present in a second syringe chamber (see e.g., U.S. Published Application No. 2004-0249339).

The GCB/IFG and GCB/IFGT compositions described herein can be packaged in a needleless syringe (see e.g., U.S. Pat. Nos. 6,406,455 and 6,939,324). Briefly, as one example, the injection device includes: a gas chamber containing a gas or a source of gas; a port which can allow for release of gas from the gas chamber; a plunger, which upon the release of gas from the gas chamber, can cause movement of at least a first piston; a first piston; a second piston; a first chamber, e.g. a chamber useful for drug storage and mixing; a piston housing, in which are disposed the first piston, the second piston and the first chamber; a displacement member which can, independent of the motive power of gas from the gas chamber, cause movement of one or both of the first and second pistons (the displacement member can be the plunger or a separate member); an orifice suitable for needleless injection in communication with the first chamber; wherein the first and second piston, are slideably disposed within the piston housing, and the displacement member, the source of gas, and the plunger are disposed such that: in a first position of the pistons, a second chamber, e.g., a fluid reservoir, is defined within the piston housing by the first piston, the piston housing and the second piston, the displacement member can move one or both of the pistons into a second position wherein the first piston is in a position such that the second chamber, which can be a fluid reservoir, is in communication with the first chamber, which can be a drug storage and mixing chamber, and the second piston is moved in the direction of the first piston, thereby decreasing the volume of the second chamber and allowing the transfer of fluid from the second chamber to the first chamber, the plunger, upon release of gas from the gas chamber, causes the first piston to move so as to decrease the volume of the first chamber allowing a substance to be expelled through the orifice and from the chamber and, e.g., to a subject.

The needleless syringe can include separate modules for a first component, e.g., a dry or liquid component, and a second component, e.g., a liquid component. The modules can be provided as two separate components and assembled, e.g., by the subject who will administer the component to himself or herself, or by another person, e.g., by an individual who provides or delivers health care. Together, the modules can form all or part of the piston housing of devices described herein. The devices can be used to provide any first and second component where it is desirable to store or provide the components separately and combine them prior to administration to a subject.

### Methods of Treatment

Any of the GCB/IFG and GCB/IFGT formulations described herein may be administered to a patient. GCB/IFG and GCB/IFGT formulations described herein are for use in methods of treatment a disorder related to a dysfunction in a GCase pathway, in particular Gaucher disease. The compositions are also used in the manufacture of a medicament for treating such disorders by the methods of treatment described herein.

The dose may be about 60 Units/kg, or 60 Units/kg, administered every other week. The dose may be about 30 Units/kg, or 30 Units/kg, administered every week. Alternatively, the dose may range from 30 to 80 Units/kg administered every other week, from 40 to 70 Units/kg administered every other week, from 50 to 80 Units/kg administered every other week, from 45 to 65 Units/kg administered every other week, from 40 to 60 Units/kg administered every other week, from 35 to 55 Units/kg administered every other week, from 30 to 50 Units/kg administered every other week, from 45 to 65 Units/kg administered every other week, from 50 to 70 Units/kg administered every other week, from 55 to 75 Units/kg administered every other week, from 60 to 80 Units/kg administered every other week, from 55 to 65 Units/kg administered every other week, from 45 to 55 Units/kg administered every other week, from 35 to 45 Units/kg administered every other week, or from 65 to 75 Units/kg administered every other week. Alternatively, the dose may range from 15 to 40 Units/kg administered every week, from 20 to 35 Units/kg administered every week, from 25 to 40 Units/kg administered every week, from 22.5 to 32.5 Units/kg administered every week, from 20 to 30 Units/kg administered every week, from 17.5 to 22.5 Units/kg administered every week, from 15 to 25 Units/kg administered every week, from 22.5 to 32.5 Units/kg administered every week, from 25 to 35 Units/kg administered every week, from 22.5 to 37.5 Units/kg administered every week, from 30 to 40 Units/kg administered every week, from 27.5 to 32.5 Units/kg administered every week, from 22.5 to 27.5 Units/kg administered every week, from 17.5 to 22.5 Units/kg administered every week, or from 32.5 to 37.5 Units/kg administered every week. Typically, the dose is 15-60 Units/kg administered every other week, in particular 60 Units/kg administered every other week. Dose adjustments can be made on an individual basis based on achievement and maintenance of therapeutic goals.

The dose may be about 1.5 mg/kg, or 1.5 mg/kg, administered every other week. The dose may be about 0.75 mg/kg, or 0.75 mg/kg, administered every week. Alternatively, the dose may range from 0.75 to 2.0 mg/kg administered every other week, from 1.0 to 1.75 mg/kg administered every other week, from 1.25 to 2.0 mg/kg administered every other week, from 1.125 to 1.625 mg/kg administered every other week, from 1.0 to 1.5 mg/kg administered every other week, from 0.875 to 1.375 mg/kg administered every other week, from 0.75 to 1.25 mg/kg administered every other week, from 1.215 to 1.625 mg/kg administered every other week, from 1.25 to 1.75 mg/kg administered every other week, from 1.375 to 1.875 mg/kg administered every other week, from 1.5 to 2.0 mg/kg administered every other week, from 1.375 to 1.625 mg/kg administered every other week, from 1.125 to 1.375 mg/kg administered every other week, from 0.875 to 1.125 mg/kg administered every other week, or from 1.625 to 1.875 mg/kg administered every other week. Alternatively, the dose may range from 0.375 to 1.0 mg/kg administered every week, from 0.5 to 0.875 mg/kg administered every week, from 0.625 to 1.0 mg/kg administered every week, from 0.5625 to 0.8125 mg/kg administered every week, from 0.5 to 0.75 mg/kg administered every week, from 0.4375 to 0.5625 mg/kg administered every week, from 0.375 to 0.625 mg/kg administered every week, from 0.5625 to 0.8125 mg/kg administered every week, from 0.625 to 0.875 mg/kg administered every week, from 0.5625 to 0.9375 mg/kg administered every week, from 0.75 to 1.0 mg/kg administered every week, from 0.6875 to 0.8125 mg/kg administered every week, from 0.5625 to 0.6875 mg/kg administered every week, from 0.4375 to 0.5625 mg/kg administered every week, or from 0.8125 to 0.9375 mg/kg administered every week. Typically, the dose is 15 mg/kg, administered every other week, in particular by subcutaneous administration. Dose adjustments can be made on an individual basis based on achievement and maintenance of therapeutic goals.

Any of the GCB/IFG and GCB/IFGT formulations described herein may be administered to a patient. The dose may be about 90 to 180 Units/kg, administered every other week. The dose may be about 90 Units/kg, or 90 Units/kg, administered every week. Alternatively, the dose may range from 90 to 150 Units/kg administered every other week, from 110 to 160 Units/kg administered every other week, from 120 to 180 Units/kg administered every other week, from 120 to 150 Units/kg administered every other week, from 90 to 120 Units/kg administered every other week, from 100 to 130 Units/kg administered every other week, from 110 to 140 Units/kg administered every other week, from 120 to 150 Units/kg administered every other week, from 130 to 160 Units/kg administered every other week, from 140 to 170 Units/kg administered every other week, or from 150 to 180 Units/kg administered every other week. Alternatively, the dose may range from 90 to 110 Units/kg administered every other week, from 100 to 120 Units/kg administered every other week, from 110 to 130 Units/kg administered every other week, from 120 to 140 Units/kg administered every other week, from 130 to 150 Units/kg administered every other week, from 140 to 160 Units/kg administered every other week, from 150 to 170 Units/kg administered every other week, or from 160 to 180 Units/kg administered every other week.

The dose may be about 2.25 to 4.5 mg/kg, administered every other week. Alternatively, the dose may range from 2.25 to 3.75 mg/kg administered every other week, from 2.75 to 4.0 mg/kg administered every other week, from 3.0 to 4.5 mg/kg administered every other week, from 3.0 to 3.75 mg/kg administered every other week, from 2.25 to 3.0 mg/kg administered every other week, from 2.5 to 3.25 mg/kg administered every other week, from 2.75 to 3.5 mg/kg administered every other week, from 3.0 to 3.75 mg/kg administered every other week, from 3.25 to 4.0 mg/kg administered every other week, from 3.5 to 4.25 mg/kg administered every other week, or from 3.75 to 4.5 mg/kg administered every other week. Alternatively, the dose may range from 2.25 to 2.75 mg/kg administered every other week, from 2.5 to 3.0 mg/kg administered every other week, from 2.75 to 3.25 mg/kg administered every other week, from 3.0 to 3.5 mg/kg administered every other week, from 3.25 to 3.75 mg/kg administered every other week, from 3.5 to 4.0 mg/kg administered every other week, from 3.75 to 4.25 mg/kg administered every other week, or from 4.0 to 4.5 mg/kg administered every other week.

Administration of the GCB/IFG and GCB/IFGT compositions can be undertaken to treat a disorder related to a dysfunction in the GCase pathway, such as lysosomal storage diseases. Exemplary lysosomal storage diseases include Gaucher disease, Fabry disease, Pompe disease, mucopolysaccharidoses, and multiple system atrophy. Compositions described herein are especially suitable for treating Gaucher disease. The disorder may be a neurodegenerative disorder, e.g., Parkinson disease, Alzheimer's disease, or Lewy body dementia. Alternatively, the disorder may involve alpha-synuclein dysregulation.

In treating the disorder, the GCB/IFG and GCB/IFGT compositions can be administered intravenously or subcutaneously. Subcutaneous administration includes subcutaneous injection, which is especially suitable for practicing the invention. Various dosing schedules may be used to administer the compositions. For example, the composition may be administered once weekly, once every two weeks, or once per month. The composition may be administered every three days, every four days, every five days, every six days, every eight days, every nine days, every 10 days, every 11 days, every 12 days, every 13 days, every 15 days, or every 16 days, for example. The frequency of administration may be changed throughout a course of treatment due to various factors. Typically, the compositions described herein are administered subcutaneously by injection either once or twice a week, or once every other week.

Where the compositions described herein are described by subcutaneous administration, care should be taken to minimize patient discomfort during administration. Therefore, typically the total volume administered to the patient per injection does not exceed 5 mL. More typically, the subcutaneously administered volume will be less than 2.5 mL per injection. If multiple subcutaneous injections are required to achieve a therapeutically effective dose, these may be administered at different sites. Alternatively, the treatment interval may be reduced. Dose adjustments can be made on an individual basis based on achievement and maintenance of therapeutic goals.

### Examples

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1: Concentration of GCB

GCB (5 ml of 10 mg/ml) was thawed after storage at −80° C. The GCB was then concentrated by centrifugal filtration at 3800 rpm, 4° C. for 30 minutes. The GCB was then diluted by 50× and the concentration measured at A280. A concentration of 100 mg/ml GCB was obtained. Then, 1% polysorbate 20 was added to a final concentration of 0.1%. To some of the solution, 20 mg of pH-adjusted isofagomine was added.

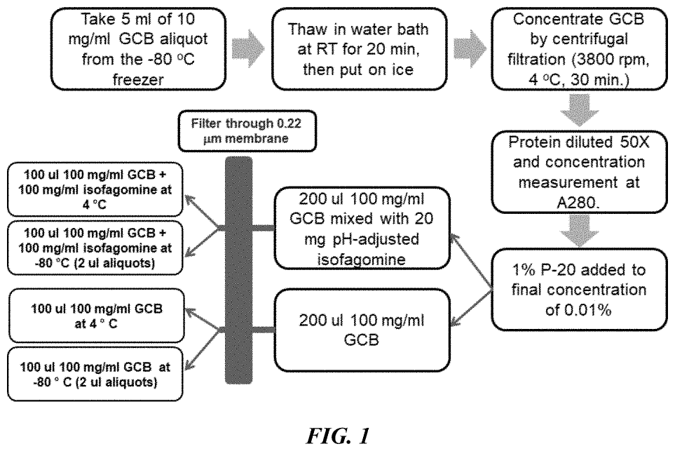

Filtration through a 0.22 um membrane was performed. A specific process is shown in FIG. 1.

### Example 2: Addition of Non-pH Adjusted IFG Destabilizes GCB

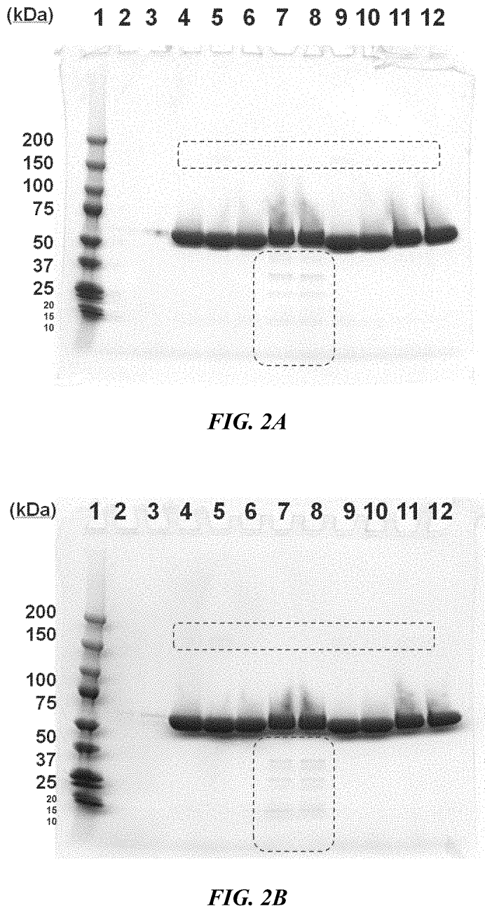

SDS-PAGE was used to analyze a variety of GCB samples, as shown below. Samples were denatured at 37° C. for 15 minutes. SDS-PAGE was run on an 8-16% Novex™ Tris-glycine pre-cast gel. 50 mM dithiothretiol was used as the reducing agent. Some of the samples have added isofagomine that was not pH-adjusted. The results from the first day are shown in FIG. 2A:
Lane 1: Molecular weight markers
Lane 2: 0.5% assay control (60 ng GCB)
Lane 3: 1% assay control (120 ng GCB)
Lane 4: 12 μg GCB Reference, reduced
Lane 5: 12 μg GCB (4° C.) Day 1, non-reduced
Lane 6: 12 μg GCB (4° C.) Day 1, reduced
Lane 7: 12 μg GCB (4° C.) Day 1 with 12 isofagomine, non-reduced
Lane 8: 12 μg GCB (4° C.) Day 1 with 12 isofagomine, reduced
Lane 9: 12 μg GCB (−80° C.) Day 1, non-reduced
Lane 10: 12 μg GCB (−80° C.) Day 1, reduced
Lane 11: 12 μg GCB (−80° C.) Day 1 with 12 isofagomine, non-reduced.
Lane 12: 12 μg GCB (−80° C.) Day 1 with 12 isofagomine, reduced

The results after two weeks are shown in FIG. 2B:
Lane 1: Molecular weight markers
Lane 2: 0.5% assay control (60 ng GCB)
Lane 3: 1% assay control (120 ng GCB)
Lane 4: 12 μg GCB Reference, reduced
Lane 5: 12 μg GCB (4° C.) Week 2, non-reduced
Lane 6: 12 μg GCB (4° C.) Week 2, reduced
Lane 7: 12 μg GCB (4° C.) Week 2 with 12 isofagomine, non-reduced
Lane 8: 12 μg GCB (4° C.) Week 2 with 12 isofagomine, reduced.
Lane 9: 12 μg GCB (−80° C.) Week 2, non-reduced
Lane 10: 12 μg GCB (−80° C.) Week 2, reduced
Lane 11: 12 μg GCB (−80° C.) Week 2 with 12 isofagomine, non-reduced
Lane 12: 12 μg GCB (−80° C.) Week 2 with 12 isofagomine, reduced.

The concentrating procedure itself may have induced cysteine-related oligomerization of GCB, as shown by faint bands from between 150 kDa to 200 kDa in lanes 4-12 that may comprise around 0.5% of total protein. Substantially more fragments of GCB were seen when isofagomine was added to GCB at 4° C. than when isofagomine was added to GCB at −80° C., as shown by several faint bands at sizes of less than 50 kDa in lanes 7 and 8 of FIG. 2A. The appearance of the faint bands may be due to the destabilization of GCB by acidic isofagomine.

Addition of isofagomine yielded fragments of GCB at 4° C. but not at −80° C. See lanes 5-8 of FIGS. 2A and 2B.

### Example 3: pH Adjustment of IFGT and Subsequent Lypophilization

When IFGT is dissolved in water, an acidic solution results. In particular, when 103 mg isofagomine tartrate was dissolved in 5 ml of water, the pH of the solution is 3.25. The pH was adjusted to 6.0 by adding 15 μl 10 M sodium hydroxide to the solution.

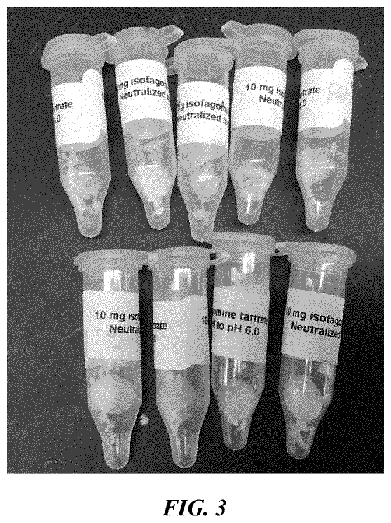

500 μl aliquots of the pH adjusted IFGT solution were added to 2 ml Eppendorf tubes. The Eppendorf tube-containing solutions were frozen on dry ice for an hour, covered by parafilm that was poked with a needle and lyophilized overnight. The Eppendorf tubes with the lyophilizates are shown in FIG. 3.

### Example 4: Addition of GCB to pH Adjusted IFG

Before adding GCB to the pH 6.0-adjusted IFGT, the pH of a GCB solution was also adjusted to 6.0 with sodium citrate. In particular, 100 mg/ml GCB in 50 mM sodium citrate yields a solution with pH 6.0. When 100 mM/ml IFGT (at pH 6.0) was added to 100 mg/ml GCB in 50 mM sodium citrate, the pH was 6.0.

### Example 5: Addition of pH Adjusted IFG does not Destabilize GCB

SDS-PAGE was used to analyze a variety of GCB samples prepared on the same day (Day 0) and after three days of storage (Day 3), as shown below, that include GCB added to pH-adjusted isofagomine. Samples were denatured at 37° C. for 15 minutes. SDS-PAGE was run on an 8-16% Novex™ Tris-glycine pre-cast gel. 50 mM dithiothretiol was used as the reducing agent. Some of the samples have added isofagomine, which was not pH-adjusted.

Lane 1: Molecular weight markers
Lane 2: 0.5% assay control (60 ng GCB)
Lane 3: 1% assay control (120 ng GCB)
Lane 4: 12 μg GCB Reference, non-reduced
Lane 5: 12 μg GCB from 100 mg/ml concentration, non-reduced
Lane 6: 12 μg GCB from 100 mg/ml concentration, reduced
Lane 7: 12 μg GCB from 100 mg/ml concentration with 12 μg pH-adjusted isofagomine, non-reduced
Lane 8: 12 μg GCB from 100 mg/ml concentration with 12 μg pH-adjusted isofagomine, reduced

SEC HPLC was performed on the Day 3 samples to confirm stability. The parameters included Gibco DPBS with addition of 400 mM sodium chloride as the mobile phase, a flow speed of 0.8 ml/min., Sepax Zenix-C SEC-150. 3 μm, 150 A, 7.8×300 mm as the SEC column, and a column temperature of 25° C.

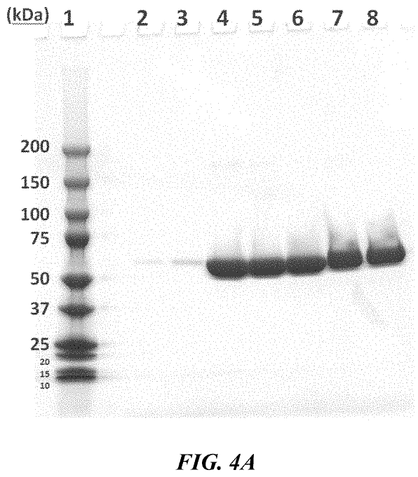

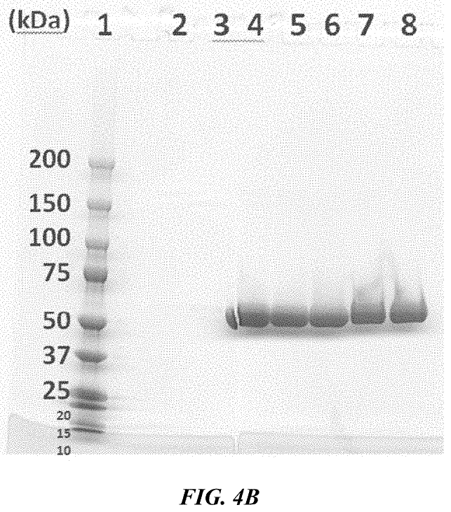

The results are shown in FIG. 4A for Day 0 and in FIG. 4B for Day 3. The GCB band was seen in lanes 4-8. No smaller bands having sizes of less than 50 kDa were observed at either Day 0 or Day 3. The adjustment of the pH of isofagomine to 6.0, which is similar to that of GCB, may minimize the destabilization of GCB.

### Example 6: Analysis of pH Adjusted IFGT on GCB Stability

GCB was concentrated to 100 mg/ml. 100 mg/ml of isofagomine tartrate (IFGT) was pH-adjusted to 6.0. The IFGT was then mixed with GCB. When pH adjustment of IFGT was not undertaken, the GCB/IFGT was not stable and protein clipping was observed by SDS-PAGE.

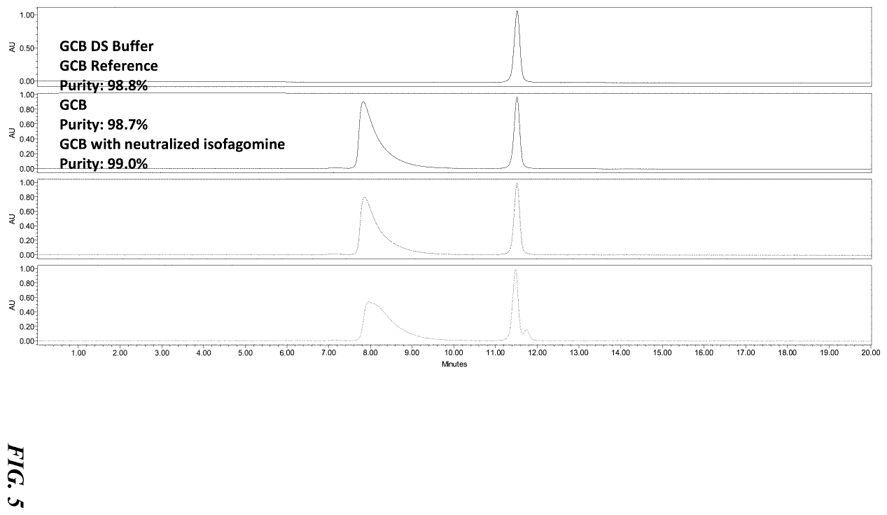

When pH adjustment was undertaken, the GCB in the solution was stable for at least three days as measured by SEC. The mobile phase was Gibco DPBS with addition of 400 mM sodium chloride, the flow speed was 0.8 ml/min, the SEC column was Sepax Zenic-C SEC-150, 3 μm, 150 A, 7.8×300 mm. The column temperature was 25° C. Four samples were analyzed by SEC, shown in FIG. 5. GCB in DS buffer, a GCB reference with 98.8% purity, and GCB with 98.7% purity were run as standards. GCB with neutralized isofagomine (pH adjusted to 6.0) that was stored for three days was also analyzed on SEC and appeared stable.

### Example 7: Size Exclusion Chromatography Analysis of pH Adjusted IFG on GCB Stability

An SEC separation assay was performed on at least the following samples listed in Table 1 below:

TABLE 1

| Sample Name | Description |
|---|---|
| GR | GCB diluted to 2.04 mg/ml |
| (GCB Reference) | |
| G4 | 4° C. GCB sample diluted to 2.01 mg/ml |
| GI4 | 4° C. GCB/IFG sample diluted to 1.81 mg/ml |
| G80 | −80° C. GCB sample diluted to 1.90 mg/ml |
| GI80 | −80° C. GCB/IFG ° C. sample diluted to 1.81 mg/ml |

The following parameters were used for SEC: Gibco DPBS with addition of 400 mM sodium chloride was used as the mobile phase. The flow speed was 0.8 ml/min. The SEC column was Sepax Zenix-C SEC-150. 3 μm, 150 A, 7.8×300 mm. The column temperature was 25° C.

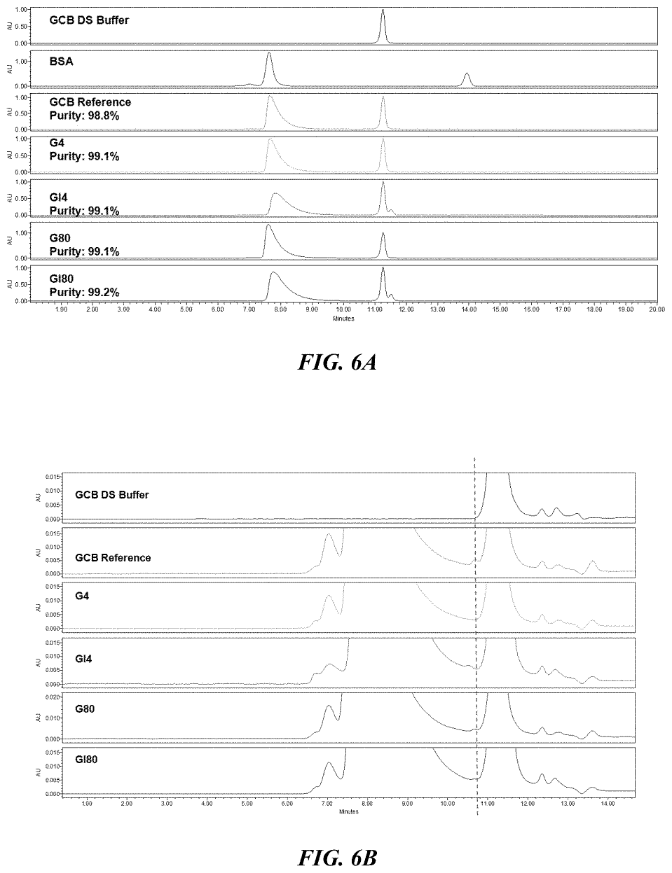

The results are shown in FIGS. 6A and 6B. The peptide fragments observed with SDS-PAGE appear in a peak eluting at about 10 minutes and 30 seconds to 10 minutes and 45 seconds. The sample at −80° C. with both isofagomine and GCB has less of a peak associated with peptide fragments than does either sample at 4° C. or even the GCB sample at −80° C.

### Example 8: Effect of GCB and IFG Concentrations on Viscosity

Various formulations of (a) GCB and (b) GCB mixed with isofagomine (GCB/IFG) were prepared. The GCB formulations included 10 mg/ml GCB, 25 mg/ml GCB, 50 mg/ml GCB, 75 mg/ml GCB, and 100 mg/ml GCB. The GCB/IFG formulations included 10 mg/ml GCB with 5 mg/mL IFG tartrate, 25 mg/ml GCB with 12.5 mg/mL IFG tartrate, 50 mg/ml GCB with 25 mg/mL IFG tartrate, 75 mg/ml GCB with 37.5 mg/mL IFG tartrate, and 100 mg/ml GCB with 50 mg/mL IFG tartrate. The viscosity and shear rate of each formulation was measured in a viscometer (m-VROC from RheoSense, San Ramon, Calif., USA). About 200 μl sized samples are needed for each measurement. The viscosity results with Slope Fit Rsqrd >0.98 are reported. The results are shown in the following tables:

TABLE 2

| | Viscosity | Shear Rate | Slope Fit |
|---|---|---|---|
| Sample | (cP) | (1/s) | R squared |
| 100 mg/ml GCB | 4.952 | 119.4 | 0.9973 |
| 75 mg/ml GCB | 2.455 | 621.4 | 0.9998 |
| 50 mg/ml GCB | 1.722 | 953.6 | 0.9999 |
| 25 mg/ml GCB | 1.312 | 1192.4 | 0.9994 |
| 10 mg/ml GCB | 1.074 | 1192.4 | 0.9999 |

TABLE 3

| | Viscosity | Shear Rate | Slope Fit |
|---|---|---|---|
| Sample | (cP) | (1/s) | R squared |
| 100 mg/ml GCB + 50 mg/ml IFGT | 4.969 | 579.2 | 0.9896 |
| 75 mg/ml GCB + 37.5 mg/ml IFGT | 2.911 | 95.2 | 0.96 |
| 50 mg/ml GCB + 25 mg/ml IFGT | 1.565 | 953.6 | 0.9998 |
| 25 mg/ml GCB + 12.5 mg/ml IFGT | 1.224 | 477.7 | 0.999 |
| 10 mg/ml GCB + 5 mg/ml IFGT | 1.064 | 1192.4 | 0.9998 |

The viscosity is correlated with the concentration of GCB. The formulation with 100 mg/ml GCB and 50 mg/ml IFG tartrate has a viscosity of approximately 5 cP, which is amenable to subcutaneous injection.

### Example 9: IFG Binding to GCB at pH 7.4 and pH 5.0 Measured by Biacore

Experiments were performed to characterize GCB and isofagomine binding affinity and kinetics at pH 7.4 and 5.0 by surface plasmon resonance (SPR). These pH may illustrate how GCB and isofagomine bind to one another in different environments, such as plasma, cytoplasm and lysosome compartments that have differing pH values.

All SPR experiments were performed on a Biacore S-200 by a single-cycle kinetics method. For experiments at pH 7.4, 2 mg/ml GCB was diluted into the GE acetate pH 5.0 buffer to the final concentration of 100 μg/ml. The immobilization running buffer was 10 mM HEPES, 5 mM EDTA, 0.01% P-20, pH 7.4. This buffer was used directly in the binding assay subsequently.

For experiments at pH 5.0, 2 mg/ml GCB was diluted into the GE acetate pH 5.0 buffer to the final concentration of 100 μg/ml. The immobilization running buffer was 20 mM sodium phosphate, 2.7 mM potassium chloride, 137 mM sodium chloride, 5 mM tartrate, 0.01% P-20, pH 5.0. Tartrate was added to the running buffer to eliminate the solute effect introduced by isofagomine tartrate. GCB was immobilized on a CM5 chip using a normal imine coupling procedure. The target immobilization level was 4000 RU.

For Biacore experiments at pH 7.4, the concentration range was 0.39-100 nM. The total was 9 points with 2-fold serial dilution. For Biacore experiments at pH 5.0, the concentration range was 0.39-100 nM. The total was 9 points with 2-fold serial dilution.

The conditions for the binding assay were as follows: 30 μl/min flow rate, 120 s association time, 600 s dissociation time, and 3 M magnesium chloride as the regeneration reagent. Isofagomine concentrations ranging initially from 0.3 μM up to 100 μM were flowed over immobilized velaglucerase alfa in single-cycle mode, without surface regeneration.

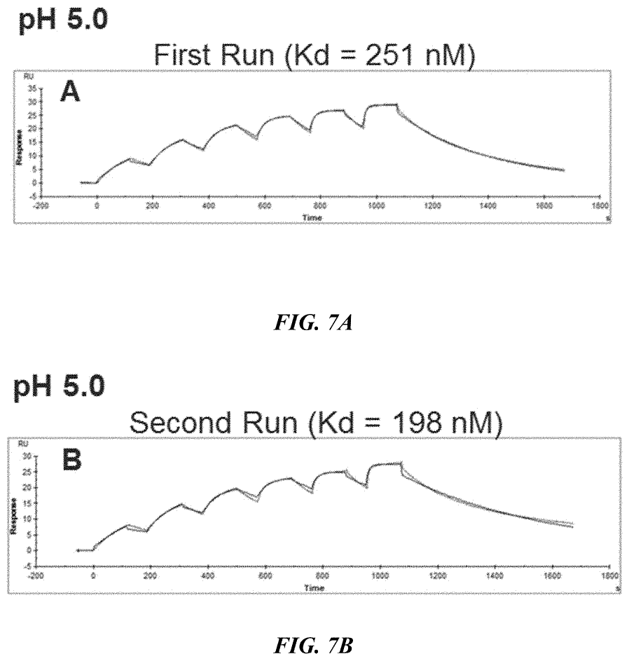

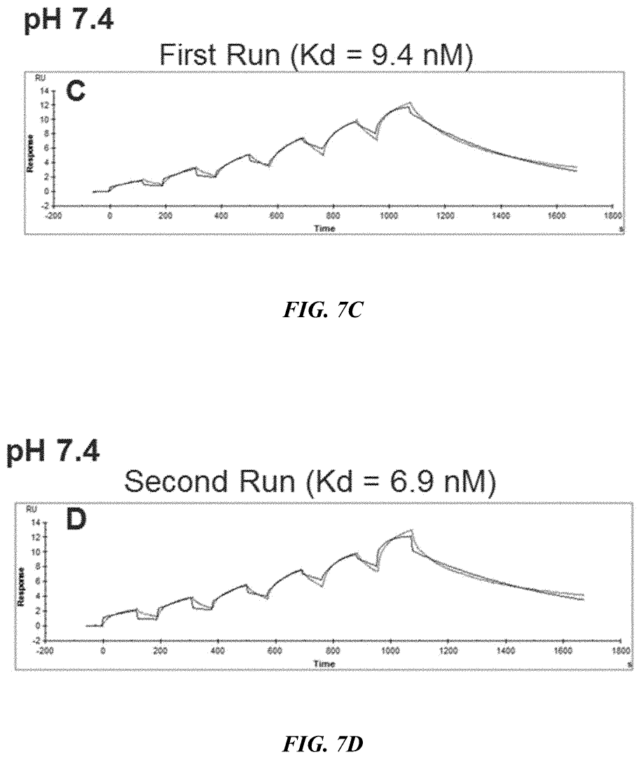

For each of the pH 5.0 and pH 7.4 studies, two runs were performed. The data obtained are shown in the following tables and in FIGS. 7A-7D. Black lines represent actual data and red lines represent model fitting.

TABLE 4
First Run at pH 5.0

| | | | Rmax | | Chi² | |
|---|---|---|---|---|---|---|
| ka (1/Ms) | kd (1/s) | K_{D} (M) | (RU) | tc | (RU²) | U-value |
| 1.25 × 10⁴ | 0.00314 | 2.51 × 10⁻⁷ | 26.73 | 8.51 × 10⁵ | 0.138 | 1 |

TABLE 5
Second Run at pH 5.0

| | | | Rmax | | Chi² | |
|---|---|---|---|---|---|---|
| ka (1/Ms) | kd (1/s) | K_{D} (M) | (RU) | tc | (RU²) | U-value |
| 9883 | 0.001954 | 1.98 × 10⁻⁷ | 24.39 | 1.70 × 10¹⁰ | 0.339 | 1 |

TABLE 6
First Run at pH 7.4:

| | | | Rmax | | Chi² | |
|---|---|---|---|---|---|---|
| ka (1/Ms) | kd (1/s) | K_{D} (M) | (RU) | tc | (RU²) | U-value |
| 2.42 × 10⁵ | 0.002274 | 9.40 × 10⁻⁹ | 12.1 | 1.08 × 10⁹ | 0.105 | 1 |

TABLE 7
Second Run at pH 7.4:

| | | | Rmax | | Chi² | |
|---|---|---|---|---|---|---|
| ka (1/Ms) | kd (1/s) | K_{D} (M) | (RU) | tc | (RU²) | U-value |
| 2.80 × 10⁵ | 0.001785 | 6.38 × 10⁻⁹ | 10.86 | 6.90 × 10⁹ | 0.178 | 2 |

The K_{D} of GCB/IFG binding at pH 5.0 is 198-251 nM. The K_{D} of GCB/IFG binding at pH 7.4 is 6.4-9.4 nM.

### Example 10: Isofagomine Increases Melting Temperature of Velaglucerase Alfa

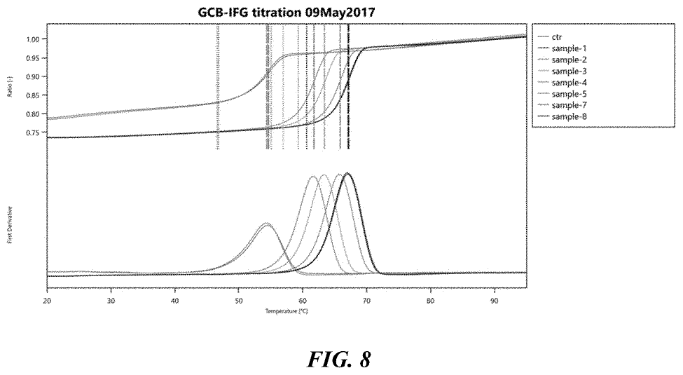

Thermal stability of velaglucerase alone or in combination with different ratios of isofagomine was evaluated using nano-differential scanning fluorimetry (nano-DSF) (FIG. 8). Samples were initially prepared at the indicated isofagomine molar ratio at a 40 mg/mL velaglucerase alfa concentration. Prior to loading onto the nano-DSF apparatus, samples were diluted down to 2 mg/mL velaglucerase alfa concentrations. The sample conditions listed in FIG. 8 are as follows:

Ctr: no isofagomine D-tartrate (IFGT)

Sample 1: 100× molar ratio IFGT

Sample 2: 30× molar ratio IFGT

Sample 3: 10× molar ratio IFGT

Sample 4: 3× molar ratio IFGT

Sample 5: no IFGT

Sample 7: 100× molar ratio isofagomine hydrochloride

Sample 8: 100× molar ratio isofagomine acetate

Isofagomine binding to velaglucerase alfa was also determined with a GCB enzyme activity assay. Enzymatic reactions were run for 1 hour at 37° C. Isofagomine tartrate was pre-incubated with velaglucerase alfa for approximately 10 minutes.

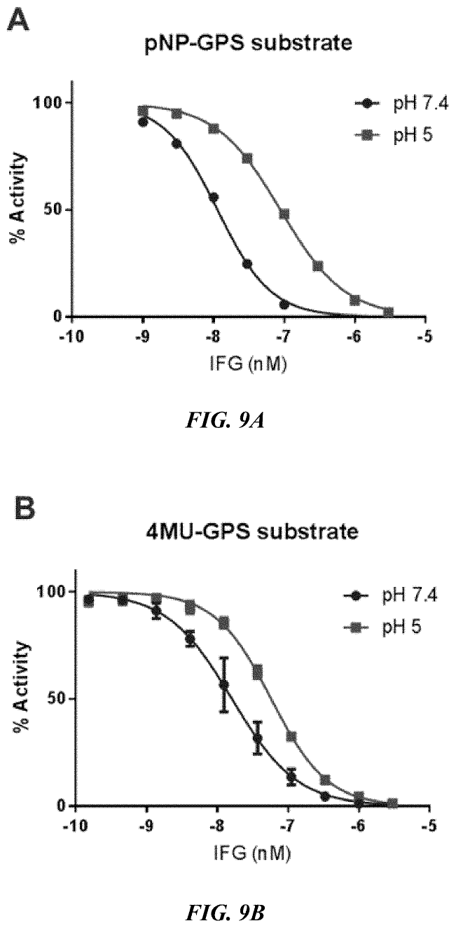

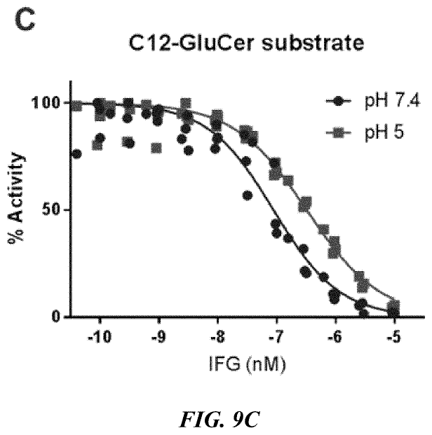

The assay concentrations for isofagomine tartrate are as indicated in the graphs shown in FIGS. 9A-9C. Final assay concentrations for velaglucerase alfa were ˜1 nM at pH 5.0 and ˜10 nM at pH 7.4. FIG. 9A shows an activity inhibition curve with synthetic colorimetric pNP-GPS substrate. FIG. 9B shows an activity inhibition curve with synthetic fluorometric 4MU-GPS substrate. FIG. 9C shows activity inhibition with natural glycosphingolipid C12-GluCer substrate. The C12-GluCer cleavage reaction was assessed by measuring glucose production with a glucose oxidase assay kit.

### Example 11: Three Week Stability Study

Four different mixtures of GCB and isofagomine D-tartrate were prepared as shown in Table 8 below.

TABLE 8

| | | IFG D-tartrate | |
|---|---|---|---|
| | GCB concentration | concentration | Molar ratio of |
| Mixture Name | (mg/ml) | (mg/ml) | GCB to IFGT |
| Group 1 | 15 | 2.25 | 1:30 |
| Group 2 | 15 | 0.75 | 1:10 |
| Group 3 | 15 | 0.225 | 1:3 |
| Group 4 | 15 | 0.075 | 1:1 |

At the initial time point and after storage for three weeks at 40° C., the specific activity and purity as measured by each of SEC, rpHPLC, and SDS-PAGE were assayed. SEC can detect soluble high-molecular weight species, while rpHPLC provides information about the chemical stability of GCB, such as resistance to oxidation. SDS-PAGE can detect protein clipping and aggregation. For specific activity, the activities of the reference standard were 16 μmol/min/mg (day 0) and 18 μmol/min/mg (week 3). Significant day-to-day variability was observed with fluorescence-based activity assays. All stability samples had slightly higher activity than that of the reference standard.

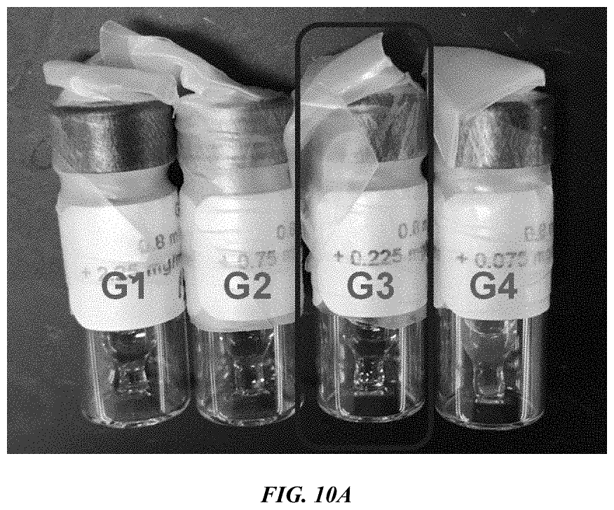

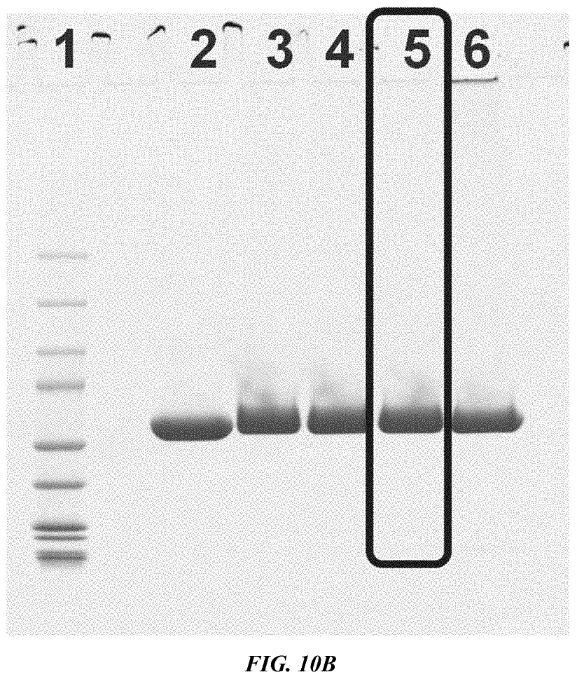

A visual inspection was also performed. Images of the samples are shown in FIG. 10A. The data are shown in the tables below. In the SDS-PAGE results shown in FIG. 10B, the following lanes correspond to the above samples:
Lane 1: Molecular weight markers
Lane 2: 12 μg GCB reference, non-reduced
Lane 3: 12 μg GCB Group 1, non-reduced
Lane 4: 12 μg GCB Group 2, non-reduced
Lane 5: 12 μg GCB Group 3, non-reduced
Lane 6: 12 μg GCB Group 4, non-reduced

TABLE 9

| | Specific activity | Specific activity |
|---|---|---|
| | (μmol/min/mg) | (mol/min/mg) |
| Mixture Name | at Day 0 | at Week 3 |
| Group 1, 1:30 molar | 21 | 28 |
| ratio of GCB to IFG | | |
| Group 2, 1:10 molar | 20 | 22 |
| ratio of GCB to IFG | | |
| Group 3, 1:3 molar | 25 | 28 |
| ratio of GCB to IFG | | |
| Group 4, 1:1 molar | 22 | 22 |
| ratio of GCB to IFG | | |

TABLE 10

| | SEC Purity (%) | SEC Purity (%) |
|---|---|---|
| Mixture Name | at Day 0 | at Week 3 |
| Group 1, 1:30 molar | 99.6 | 99.4 |
| ratio of GCB to IFG | | |
| Group 2, 1:10 molar | 99.5 | 99.3 |
| ratio of GCB to IFG | | |
| Group 3, 1:3 molar | 99.5 | 99.2 |
| ratio of GCB to IFG | | |
| Group 4, 1:1 molar | 99.5 | 98.8 |
| ratio of GCB to IFG | | |

TABLE 11

| | rpHPLC Purity (%) | rpHPLC Purity (%) |
|---|---|---|
| Mixture Name | at Day 0 | at Week 3 |
| Group 1, 1:30 molar | 97.4 | 96.8 |
| ratio of GCB to IFG | | |
| Group 2, 1:10 molar | 97.4 | 98.3 |
| ratio of GCB to IFG | | |
| Group 3, 1:3 molar | 97.4 | 98.1 |
| ratio of GCB to IFG | | |
| Group 4, 1:1 molar | 97.3 | 98.3 |
| ratio of GCB to IFG | | |

TABLE 12

| | SDS-PAGE Purity | SDS-PAGE Purity |
|---|---|---|
| Mixture Name | (%) at Day 0 | (%) at Week 3 |
| Group 1, 1:30 molar | >98 | >98 |
| ratio of GCB to IFG | | |
| Group 2, 1:10 molar | >98 | >98 |
| ratio of GCB to IFG | | |
| Group 3, 1:3 molar | >98 | >98 |
| ratio of GCB to IFG | | |
| Group 4, 1:1 molar | >98 | Aggregates |
| ratio of GCB to IFG | | observed |

The molar ratio of 1:1 GCB to IFG was too low to provide for stability over three weeks at 40° C. Aggregates were seen in the SDS-PAGE assay and the solution appeared cloudy at three weeks. However, at molar ratios of 1:3 GCB to IFG and above at three weeks, the purity was at least 98% in SDS-PAGE and the solutions appeared transparent.

### Example 12: Pharmacokinetic Study of Intravenous GCB and Subcutaneous GCB with IFG in the Cynomolgus Monkey

Two groups of cynomolgus monkeys were tested for the pharmacokinetics of GCB. In Group 1, the GCB was administered once by intravenous injection. In Group 2, a formulation of GCB with IFG was administered once by subcutaneous injection. Three samples from the liver and spleen were collected at each of 1 hour, 2 hours, 8 hours and 24 hours post-dose. Additional detail of the study design is shown Table 13 below:

TABLE 13

| Group Number and | Test Article and | Concentration | Dose volume | |
|---|---|---|---|---|
| Origin | Dose | (mg/ml) | (ml/kg) | ROA/TOA |
| 1 | 10 mg/kg GCB | 10 | 1 | IV injection once |
| (12 males, PNN | | | | on day 1 (T = 0) |
| origin) | | | | |
| 2 | 10 mg/kg GCB and | 100 (GCB) | 0.1 | SC injection once |
| (12 males, PNN | 5 mg/kg isofagomine | 50 (isofagomine | | on day 1 (T = 0) |
| origin) | tartrate formulated | tartrate) | | |
| | together (100x | | | |
| | IFG molar ratio) | | | |

Collections at liver and spleen made 1, 2, 8 and 24 hours (n=3) post-dose.

Histological analysis was then performed on all of the samples. 10% NBF fixed liver and spleen were processed for paraffin block. 5 micron sections were prepared for GCB IHC (primary antibody TK36-mouse anti-huGCB at 1:10,000) and Haemotoxylin and Eosin staining.

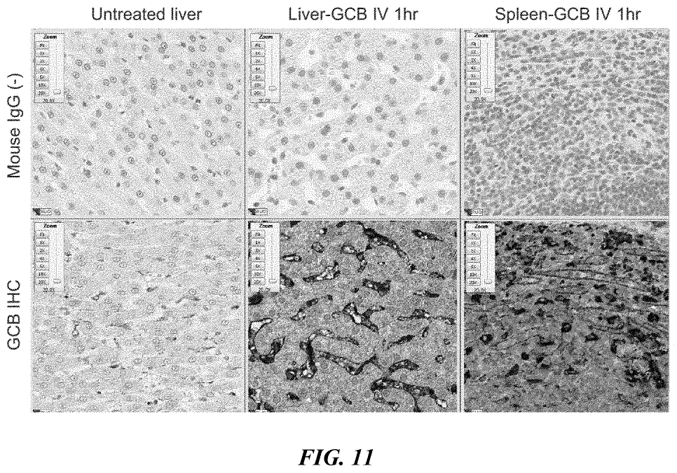

FIG. 11 shows the negative and positive controls for GCB IHC staining on monkey tissues in liver and spleen. Negligible staining was seen in absence of the GCB IHC antibody (top panels). In the presence of the GCB IHC antibody, faint background staining was seen in the untreated liver (lower left panel). Dark staining was seen in the treated liver and spleen in the presence of the GCB IHC antibody (lower middle and lower right panels). In particular, the liver showed GCB-positive staining in Kupffer cells, endothelium and hepatocytes and the spleen showed endothelium and macrophage positive staining.

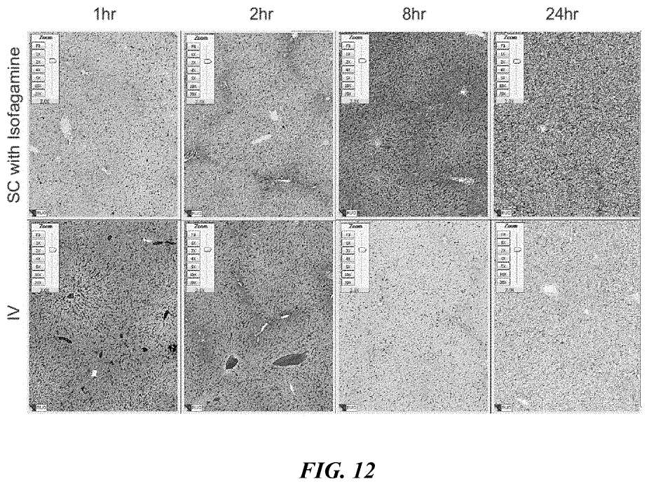

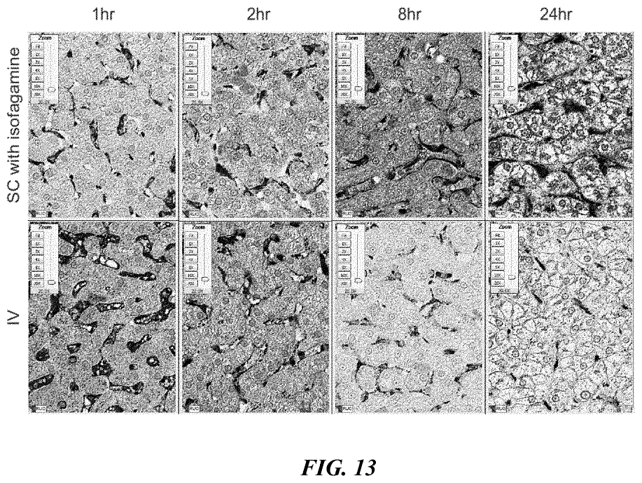

The biodistribution of GCB in the liver was studied post delivery of GCB by intravenous injection and of GCB with IFG by subcutaneous injection. In the liver of a monkey treated by subcutaneous injection, strong GCB was seen at the 8 hour time point. Strong GCB staining was seen at the one and two hour time points in the liver of a monkey treated by intravenous injection. The results are shown in FIG. 12 (2× magnification) and FIG. 13 (20× magnification).

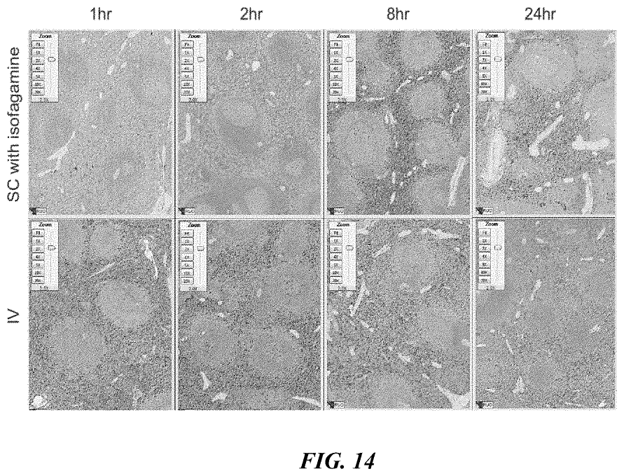

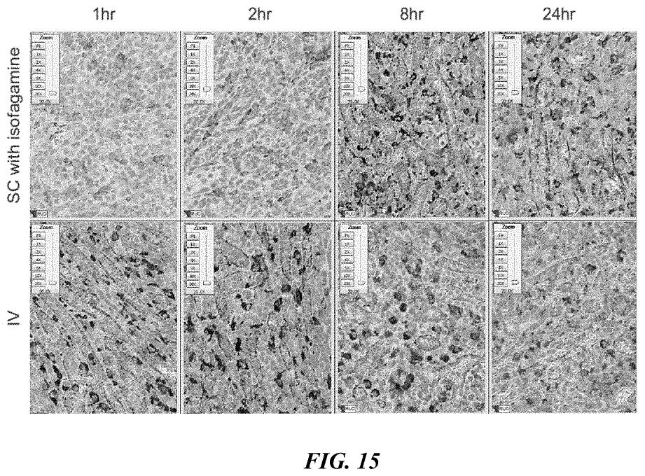

Similar results were seen in the spleen, as shown in FIG. 14 (2× magnification) and FIG. 15 (20× magnification). These data suggest that subcutaneous administration of GCB with IFG can provide comparable GCB tissue exposure to that of IV administration of GCB.

### Example 13: Correlation of Velaglucerase Alfa Activity with Protein Level in Liver and Spleen

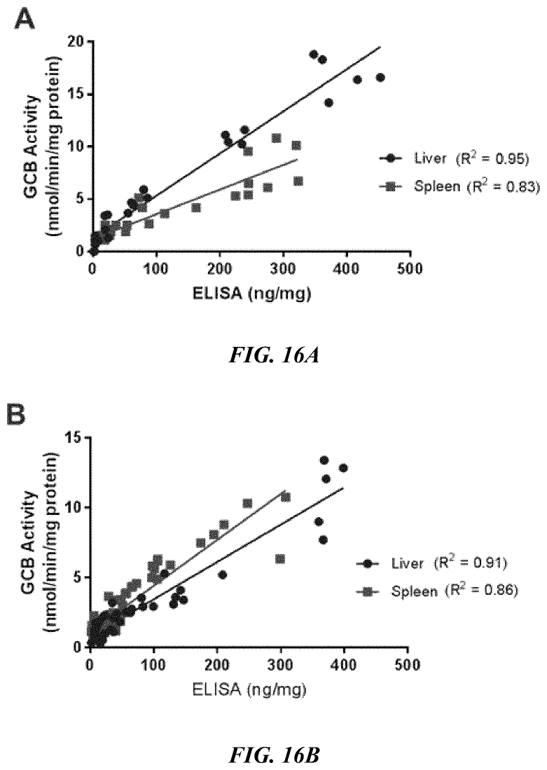

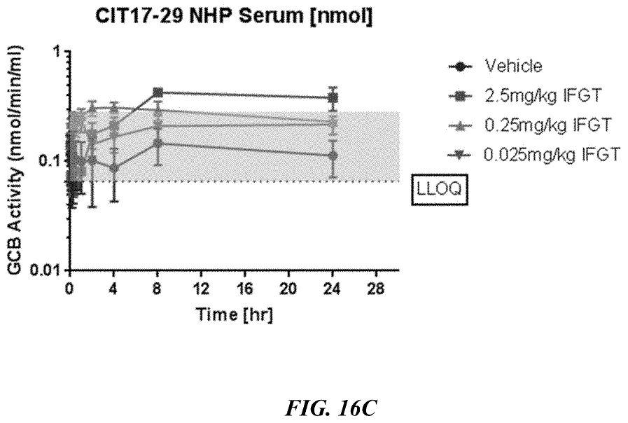

Velaglucerase alfa protein and enzyme activity levels were assessed in liver and spleen homogenates after IV dose administration in cynomolgus monkeys. Tissues were collected at pre-determined time points after dosing (0.5-24 hours). The data are shown in FIGS. 16A and 16B. In particular, FIG. 16A shows the results from IV dosing with velaglucerase alfa only over a range of 2-10 mg/kg. FIG. 16B shows the results from SC dosing with velaglucerase alfa over a range of 1.5-10 mg/kg formulated with a corresponding amount of isofagomine (0.0075-5 mg/kg) such that the molar ratio of velaglucerase alfa to isofagomine is 1:3.

### Example 14: Serum GCB Activity Levels in Cynomolgus Monkeys after Subcutaneous Administration of Isofagomine Tartrate

The serum activity levels of GCB were assayed in cynomolgus monkeys after subcutaneous (SC) administration of velaglucerase alfa with isofagomine tartrate. The data are shown in FIG. 16C. Endogenous GCB serum activity was determined from the vehicle and pre-dose animals (n=39) treated with GCB and ranged from 4-14 ng/mL or 0.07-0.25 nmol 4MU/min/mL. Isofagomine tartrate can increase GCB activity in the serum above the upper limit of normal with a SC dose of 2.5 mg/kg. This increase in serum activity is likely due to the prevention of native GCB degradation processes continuously occurring in the serum. The isofagomine tartrate dose incorporated in Vela-3×IFGT (0.0225 mg/kg) would not increase endogenous serum GCB activity, based on the data from the higher 0.025 mg/kg dose.

### Example 15: IFG Provides >25× Enhancement in Velaglucerase Alfa S C Serum Exposure

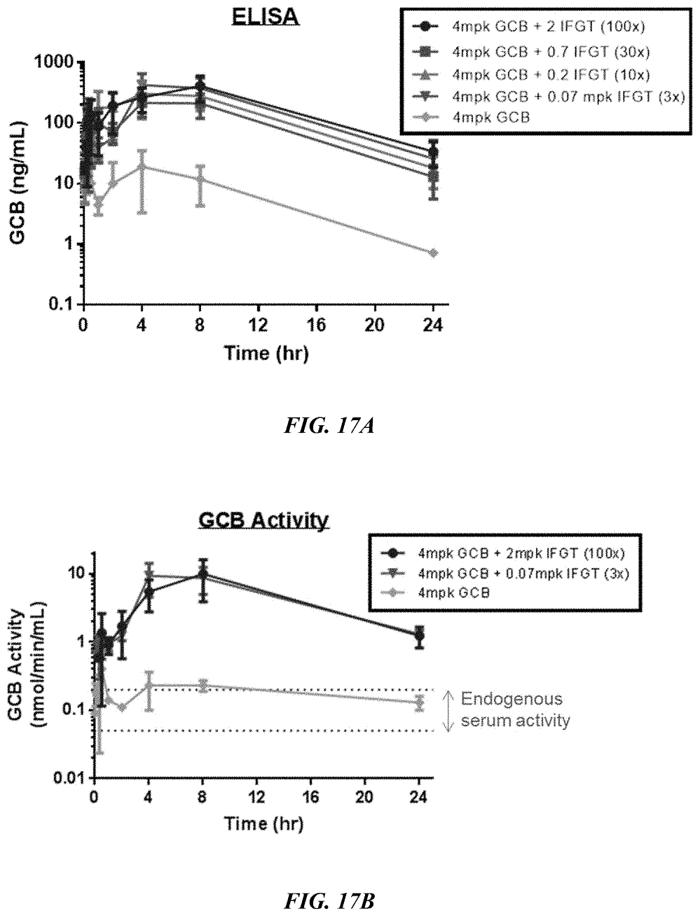

Velaglucerase alfa and IFG in a 1:3 molar ratio administered subcutaneously to cynomolgus monkeys at a 4 mg/kg dose was able to provide greater than 25-fold improvement in serum exposure compared to a 4 mg/kg IV dose of velaglucerase alfa. IFG ratios of 3-fold to 100-fold molar excess over velaglucerase alfa promoted similar increases in serum exposure. The increase in serum bioavailability as determined from the ECL ELISA assay was corroborated with the GCB activity assay (4MU-GPS substrate). The results are shown in FIGS. 17A and 17B. Addition of 0.07 mg/kg of IFGT to 4 mg/kg GCB substantially increased the amount of GCB in serum (16A) as well as the overall enzyme activity of GCB (16B). The data therefore demonstrate that when GCB is co-formulated with IFG, e.g., IFGT, particularly in a molar ratio of at least 1:3 (GCB:IFG, e.g., GCB:IFGT), it can be provided for serum bioavailability that allows for SC administration.

### Example 16: Superior Tissue Biodistribution of Subcutaneous VPRIV with IFG in a 1:100 Molar Ratio Compared to Intravenous Dosing of VPRIV Alone

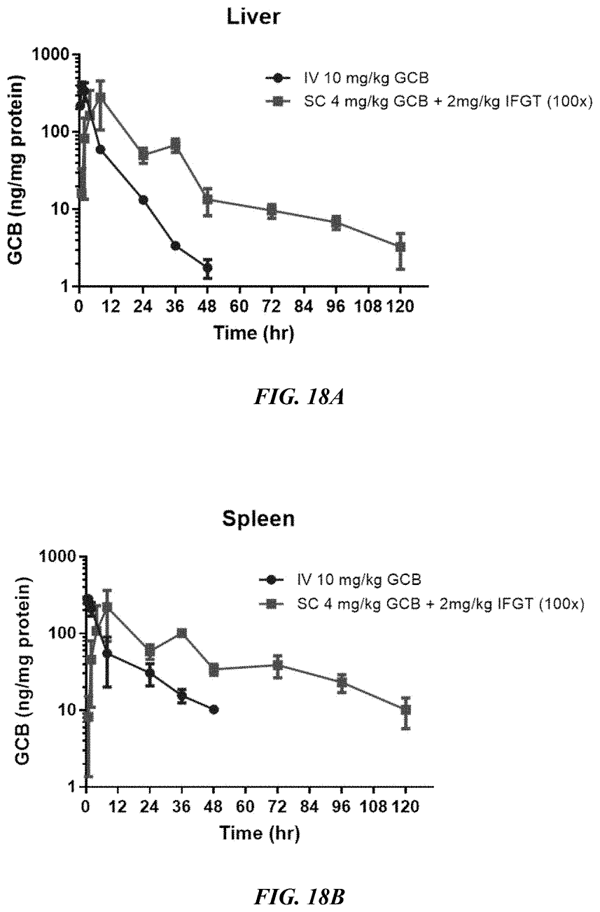

Velaglucerase alfa and IFG in a 1:100 molar ratio administered subcutaneously to cynomolgus monkeys at a 4 mg/kg dose was able to confer tissue uptake of velaglucerase alfa which exceeded that of a 10 mg/kg IV dose of velaglucerase alfa alone. Standard of care IV-infusion dosing of VPRIV is 1.5 mg/kg. In some embodiments, a target subcutaneous dose of approximately 1.5 mg/kg may be used. About 250 mg of tissue was homogenized in 1 ml of HEPES/Triton X-100 lysis buffer. Velaglucerase alfa content in the tissues was measured using the ECL ELISA assay and normalized to total protein content as determined from a BCA assay. The results are shown in FIGS. 18A and 18B. The exposure profile of GCB present in the liver (18A) and spleen (18B) after subcutaneous administration of a 1:100 molar ratio of GCB:IFG was superior that of intravenous administration of GCB over the course of 5 days (120 hours).

### Example 17: Tissue Biodistribution Comparability of Subcutaneous VPRIV with IFG in a 1:3 Molar Ratio to an Intravenous Dose of VPRIV Alone

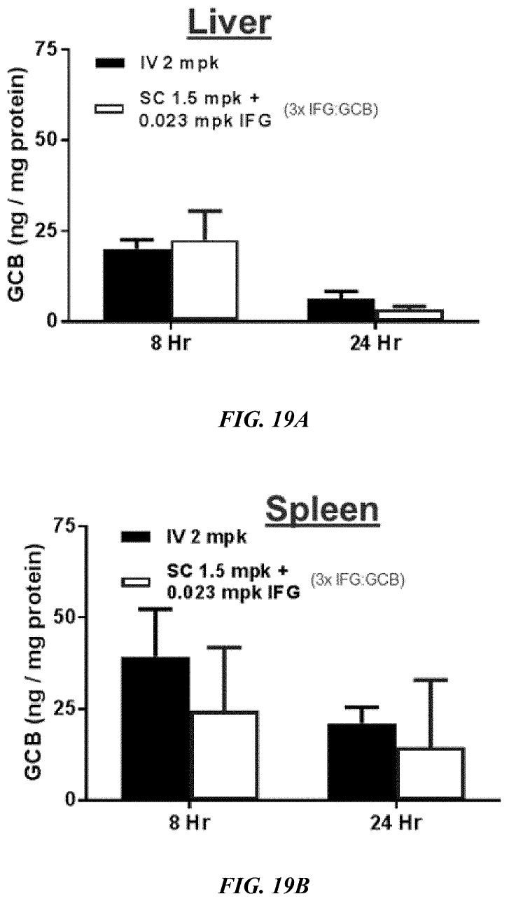

Velaglucerase alfa and IFG in a 1:3 molar ratio administered subcutaneously to cynomolgus monkeys at a target 1.5 mg/kg clinical dose was able to confer tissue uptake of velaglucerase alfa comparable to that of a 2 mg/kg IV dose of velaglucerase alfa alone. Standard of care IV-infusion dosing of VPRIV is 1.5 mg/kg. About 250 mg of tissue was homogenized in 1 ml of HEPES/Triton X-100 lysis buffer. Velaglucerase alfa content in the tissues was measured using the ECL ELISA assay and normalized to total protein content as determined from a BCA assay. The results are shown in FIGS. 19A and 19B. The amount of GCB present in the liver after subcutaneous administration of a 1:3 molar ratio of GCB:IFG was comparable to that of intravenous administration of GCB at both 8 hour and 24 hour time points. Similarly, GCB tissue exposure in the spleen after subcutaneous administration of a 1:3 molar ratio of GCB:IFG was comparable that of intravenous administration of GCB at both 8 hour and 24 hour time points.

Similarly, GCB tissue exposure in the spleen after subcutaneous administration of a 1:3 molar ratio of GCB:IFG was comparable that of intravenous administration of GCB at both 8 hour and 24 hour time points. Thus, addition of IFG, e.g., IFGT, to a GCB formulation can allow for subcutaneous administration of GCB-containing formulations.

### Example 18: Isofagomine Ratios as Low as 1:1 Provide Similar Serum Exposures as Higher Isofagomine Molar Ratios

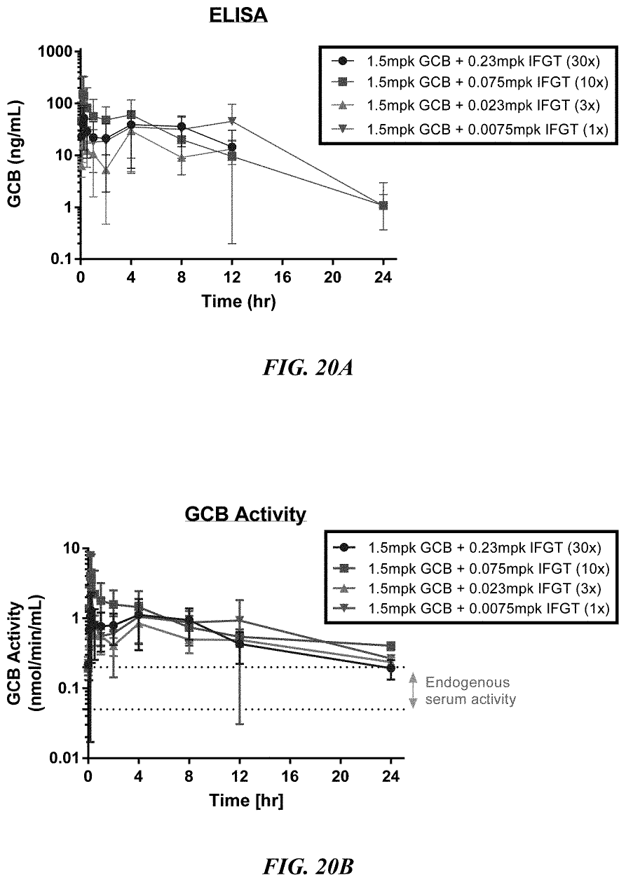

Velaglucerase alfa and IFG in a 1:1 molar ratio administered subcutaneously to cynomolgus monkeys at a 1.5 mg/kg dose was able to provide similar GCB serum exposures as higher isofagomine ratios. No obvious differences in GCB serum exposures were observed for molar ratios between 1:1 and 1:100. The increase in serum bioavailability as determined from the ECL ELISA assay was corroborated with the GCB activity assay (4MU-GPS substrate). The results are shown in FIGS. 20A and 20B.

Test articles for dosing were prepared as frozen formulations to the animal facility prior to dosing. Test articles were thawed approximately 1 to 3 hours prior to dosing. The data therefore demonstrate that if room temperature storage liabilities can be circumvented by cold temperature storage (e.g., frozen) that when GCB is co-formulated with IFG, e.g., IFGT, particularly in a molar ratio of at least 1:1 (GCB:IFG, e.g., GCB:IFGT), it can provide sufficient serum bioavailability that allows for SC administration.

### Example 19: Isofagomine Protects VPRIV Against Thermal Denaturation at 37° C. in Human Serum

Serum that contained 10 nM VPRIV (a form of GCB) was tested to determine if IFG could stabilize the GCB. IFG was added to VPRIV such that IFG had the following concentrations of IFG in the serum: 1 nM, 3 nM, 10 nM, 30 nM, 100 nM, 300 nM, and 1000 nM. A negative control was used with no added IFG.

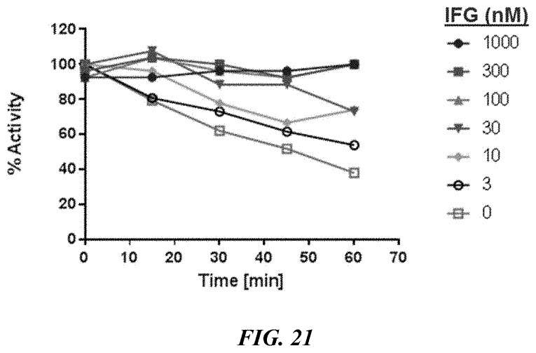

Enzyme activity was measured using the cleavage of the 4-methylumbelliferone b-D-glucopyranoside substrate. The activity diminished from 100% down to around 40% over 60 minutes with the negative control, 1 nM IFG and 10 nM IFG. See FIG. 21. However, addition of concentrations of 30 nM (3× molar ratio) and higher prevented most of the loss of activity. IFG may be effective to protect GCB against heat denaturation in serum. IFG- and IFGT-mediated protection of GCB against thermal degradation may enhance GCB bioavailability, enhance GCB persistence in serum, and enable a longer duration for cell and tissue uptake processes of GCB to occur.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

All publications, patent applications, patents, and other references mentioned herein are incorporated by reference in their entirety. In the case of conflict, the present specification, including definitions, controls. In addition, the materials, methods, and examples are illustrative only and not intended to be limiting.

1-77. (canceled)

78. A composition comprising a glucocerebrosidase (GCB) and an isofagomine (IFG) in a molar ratio of at least about 1:2.5.

79. The composition of claim 78, wherein the GCB is velaglucerase alfa.

80. The composition of claim 78, wherein the pH of the composition is about 6.0, about 6.5, or about 7.0.

81. The composition of claim 78, wherein the molar ratio of the GCB to the IFG is (a) from about 1:2.5 to about 1:30, or (b) from about 1:2.5 to about 1:10, or (c) from about 1:10 to about 1:30, or (d) about 1:2.5 to about 1:3.5, or (e) about 1:3.0, or (f) 1:3.0.

82. The composition of claim 78, wherein the composition is at a temperature of (a) at least 20° C., or (b) 0° C. to 20° C., or (c) less than 0° C.

83. The composition of claim 78, wherein the composition is a liquid or a lyophilizate.

84. The composition of claim 78, further comprising a pharmaceutically acceptable excipient, a pharmaceutically acceptable salt, or both a pharmaceutically acceptable excipient and a pharmaceutically acceptable salt.

85. The composition of claim 78, wherein the IFG is isofagomine tartrate, wherein optionally the isofagomine tartrate is isofagomine D-tartrate.

86. The composition of claim 78, further comprising an antioxidant, and/or a carbohydrate, and/or a surfactant.

87. The composition of claim 78, wherein the composition comprises 45-120 mg/mL of GCB and 0.2 to 1.8 mg/mL isofagomine, particularly 60 mg/mL of GCB and 0.9 mg/mL isofagomine.

88. The composition of claim 87, wherein the composition further comprises
a) 50 mM sodium citrate or sodium phosphate, and 0.01% polysorbate 20, or
b) 5-20 mM sodium citrate and 0.01% polysorbate-20, or
c) 10 mM sodium citrate and 0.01% polysorbate-20, or
d) 5-20 mM sodium phosphate and 0.01% polysorbate-20, or
e) 10 mM sodium phosphate and 0.01% polysorbate-20.

89. A container comprising the composition of claim 78, wherein optionally the container is selected from the group consisting of a prefilled syringe, a vial, and ampoule.

90. A method of preparing a composition of claim 78, the method comprising dissolving the IFG in water, adjusting the pH to about 6.0, and adding the GCB to yield the composition.

91. The method of claim 90, further comprising one or more of the following steps:
a) lyophilizing the IFG before adding GCB,
b) adding polysorbate 20 to 0.01%, and
c) filtering the composition through a 0.22 μm membrane.

92. The method of claim 90, wherein the IFG is present in an amount sufficient to maintain the stability of the GCB in the composition, particularly for at least three days at 0-50° C., or at least 6 months at 0-40° C.

93. A method of treating a disorder related to a dysfunction in a GCase pathway comprising administering the composition of claim 78 to a patient in need thereof.

94. The method of claim 93, wherein the composition is administered intravenously or subcutaneously, wherein optionally the subcutaneous administration is subcutaneous injection.

95. The method of claim 93, wherein the composition is administered
a) twice weekly, or
b) once weekly, or
c) less often than once weekly, or
d) once every other week.

96. The method of claim 93, wherein said disorder comprises a defect in GCase activity, wherein optionally said defect in GCase activity comprises a decreased enzymatic activity.

97. The method of claim 93, wherein said disorder comprises alpha-synuclein dysregulation.

98. The method of claim 93, wherein said disorder is a lysosomal storage disease.

99. The method of claim 98, wherein said lysosomal storage disease is selected from Gaucher disease, Fabry disease, Pompe disease, a mucopolysaccharidoses, and multiple system atrophy.

100. The method of claim 93, wherein said disorder is a neurodegenerative disorder.

101. The method of claim 100, wherein said neurodegenerative disorder is selected from Parkinson disease, Alzheimer's disease, and Lewy body dementia.

102. A method of treating a dysfunction in a GCase pathway comprising administering to a subject a composition comprising from 0.5 to 5.0 mg/kg GCB and IFG in at least about a 3-fold molar excess to the GCB, wherein the composition is administered subcutaneously.

103. The method of claim 102, wherein the composition comprises
a) from 0.8 to 4.0 mg/kg GCB, or
b) from 1.0 to 3.0 mg/kg GCB, or
c) from 1.2 to 2.0 mg/kg GCB, or
d) about 1.5 mg/kg GCB, or
e) 1.5 mg/kg GCB, or
f) from 2.0 to 5.0 mg/kg GCB, or
g) from 2.25 to 4.5 mg/kg GCB, or
h) from 2.25 to 3.75 mg/kg GCB, or
i) from 3.5 to 5.0 mg/kg GCB.

104. The method of claim 103, wherein the IFG is in
a) a 3 to 10-fold molar ratio to the GCB, or
b) a 10 to 30-fold molar ratio to the GCB, or
c) a 30 to 100-fold molar ratio to the GCB, or
d) a 3-fold molar ratio to the GCB.

105. The method of claim 93, wherein exposure, activity, or bioavailability of the GCB in the spleen, and/or liver, and/or serum is increased.
